(19) United States
(12) Patent Application Publication
(10) Pub. No.: US 2018/0185428 A1
(43) Pub. Date: Jul. 5, 2018

(54) Herbal Combinations for Wound Healing in Fibroblasts

(71) Applicant: Sirbal Ltd., Limassol (CY)

(72) Inventors: Nadav Shraibom, Herzelia (IL); Anu T. Singh, Noida (IN); Manu Jaggi, DLF City (IN); Eran Steinberg, San Francisco, CA (US)

(21) Appl. No.: 15/905,794

(22) Filed: Feb. 26, 2018

Related U.S. Application Data
(63) Continuation-in-part of application No. PCT/US17/24378, filed on Mar. 27, 2017.
Application No. PCT/US17/24378 is a continuation-in-part of application No. 15/219,394, filed on Jul. 26, 2016.
Application No. 15/219,394 is a continuation-in-part of application No. 15/219,447, filed on Jul. 26, 2016.
Application No. 15/219,447 is a continuation-in-part of application No. 15/131,743, filed on Apr. 18, 2016.
Application No. 15/131,743 is a continuation-in-part of application No. 14/754,266, filed on Jun. 29, 2015.
Application No. 14/754,266 is a continuation-in-part of application No. 13/900,525, filed on May 22, 2013, now Pat. No. 9,095,606.
Application No. 13/900,525 is a continuation-in-part of application No. 13/361,978, filed on Jan. 31, 2012.
Application No. 13/361,978 is a continuation-in-part of application No. 13/152,039, filed on Jun. 2, 2011.
Application No. 13/152,039 is a continuation-in-part of application No. 15/133,056, filed on Apr. 19, 2016.
Application No. 15/133,056 is a continuation-in-part of application No. 15/131,743, filed on Apr. 18, 2016.
Application No. 15/131,743 is a continuation-in-part of application No. 14/754,266, filed on Jun. 29, 2015.
Application No. 14/754,266 is a continuation-in-part of application No. 13/900,525, filed on May 22, 2013, now Pat. No. 9,095,606.
Application No. 13/900,525 is a continuation-in-part of application No. 13/361,978, filed on Jan. 31, 2012.
Application No. 13/361,978 is a continuation-in-part of application No. 13/152,039, filed on Jun. 2, 2011.
Application No. 13/152,039 is a continuation-in-part of application No. 14/754,266, filed on Jun. 29, 2015.
Application No. 14/754,266 is a continuation-in-part of application No. 13/900,525, filed on May 22, 2013, now Pat. No. 9,095,606.
Application No. 13/900,525 is a continuation-in-part of application No. 13/361,978, filed on Jan. 31, 2012.
Application No. 13/361,978 is a continuation-in-part of application No. 13/152,039, filed on Jun. 2, 2011.
(60) Provisional application No. 62/325,993, filed on Apr. 21, 2016.
(60) Provisional application No. 62/313,709, filed on Mar. 26, 2016.
(60) Provisional application No. 62/198,637, filed on Jul. 29, 2015.
(60) Provisional application No. 62/297,796, filed on Feb. 19, 2016.
(60) Provisional application No. 62/348,762, filed on Jun. 10, 2016.
(60) Provisional application No. 62/198,637, filed on Jul. 29, 2015.
(60) Provisional application No. 62/297,796, filed on Feb. 19, 2016.
(60) Provisional application No. 62/348,762, filed on Jun. 10, 2016.
(60) Provisional application No. 62/198,637, filed on Jul. 29, 2015.
(60) Provisional application No. 62/297,796, filed on Feb. 19, 2016.
(60) Provisional application No. 62/198,637, filed on Jul. 29, 2015.
(60) Provisional application No. 62/297,796, filed on Feb. 19, 2016.

(51) Int. Cl. A61K 36/355; A61P 17/02; A61K 36/708; A61K 36/64; A61K 9/00; A61K 9/06

(52) U.S. Cl. CPC A61K 36/355; A61P 17/02; A61K 9/06; A61K 36/64; A61K 9/0014; A61K 36/708

(57) ABSTRACT

A dosage composition for enhancing skin wound healing efficiency includes a fibroblast cell migration enhancing active component that includes at least 60 wt. % of an herbal combination consisting essentially of between three and seven herbs at least including rheum tanguticum, lonicera japonica, and rehmannia glutinosa.

### PRIORITY AND RELATED APPLICATIONS

This application is a continuation-in-part (CIP) of Patent Cooperation Treaty (PCT) application serial no. PCT/US17/24378, filed Mar. 27, 2017; which claims priority to U.S. provisional patent applications Ser. Nos. 62/325,993, filed Apr. 21, 2016; and U.S. Ser. No. 62/313,709, filed Mar. 26, 2016.

This application is a continuation-in-part (CIP) of U.S. patent application Ser. No. 15/219,394, filed Jul. 26, 2016, which claims priority to U.S. Ser. No. 62/198,637, filed Jul. 29, 2015; U.S. Ser. No. 62/297,796, filed Feb. 19, 2016; and U.S. Ser. No. 62/348,762, filed Jun. 10, 2016.

This application is a continuation-in-part (CIP) of U.S. patent application Ser. No. 15/219,447, filed Jul. 26, 2016, which claims priority to U.S. Ser. No. 62/198,637, filed Jul. 29, 2015; U.S. Ser. No. 62/297,796, filed Feb. 19, 2016; and U.S. Ser. No. 62/348,762, filed Jun. 10, 2016.

This application is a continuation-in-part (CIP) of U.S. patent application Ser. No. 15/131,743, filed Apr. 18, 2016, which claims priority to U.S. provisional patent applications Nos. 62/198,637, filed Jul. 29, 2015 and 62/297,796, filed Feb. 19, 2016; and which is a continuation-in-part (CIP) of U.S. patent application Ser. No. 14/754,266, filed Jun. 29, 2015, which is a continuation-in-part (CIP) which claims priority to U.S. patent application Ser. No. 13/900,525, filed May 22, 2013, now U.S. Pat. No. 9,095,606, which is a continuation-in-part (CIP) of U.S. Ser. No. 13/361,978, filed Jan. 31, 2012, which is a continuation-in-part (CIP) of U.S. Ser. No. 13/152,039, filed Jun. 2, 2011.

This application is a continuation-in-part (CIP) of U.S. patent application Ser. No. 15/133,056, filed April 19, 2016, which claims priority to U.S. provisional patent applications Nos. 62/198,637, filed Jul. 29, 2015 and 62/297,796, filed Feb. 19, 2016; and which is a continuation-in-part (CIP) of U.S. patent application Ser. No. 15/131,743, filed Apr. 18, 2016, and which is a continuation-in-part (CIP) of U.S. patent application Ser. No. 14/754,266, filed Jun. 29, 2015, which is a continuation-in-part (CIP) of Ser. No. 13/900,525, filed May 22, 2013, now U.S. Pat. No. 9,095,606, which is a continuation-in-part (CIP) of U.S. Ser. No. 13/361,978, filed Jan. 31, 2012, which is a continuation-in-part (CIP) of U.S. Ser. No. 13/152,039, filed Jun. 2, 2011.

This application is a continuation-in-part (CIP) of U.S. patent application Ser. No. 14/754,266, filed Jun. 29, 2015, which is a continuation-in-part (CIP) of U.S. Ser. No. 13/900,525, filed May 22, 2013, now U.S. Pat. No. 9,095,606, which is a continuation-in-part (CIP) of U.S. Ser. No. 13/361,978, filed Jan. 31, 2012, which is a continuation-in-part (CIP) of U.S. Ser. No. 13/152,039, filed Jun. 2, 2011.

This application is related to U.S. patent applications Ser. Nos. PCT/US17/39177, PCT/US16/63706, PCT/US16/43963, PCT/US15/38341, Ser. Nos. 14/815,892, 13/890,990, PCT/US13/72670, Ser. Nos. 14/815,705, 13/152,039, PCT/US11/60501, and to US published patent applications nos. 20170049688A1, 20160375076A1, 20160375077A1, 20160113982A1, 20170049687A1, 20170049835A1, 20160184380A1, 20160184379A1, 20160184381A1, 20160175372A1, 20160175371A1, 20160051553A1, 20160051553A1, 20160136220A1, 20160136219A1, 20160136216A1, 20160136223A1, 20160136222A1, 20160136221A1, 20160113983A1, 20160143980A1, 20160136218A1, 20140205685A1, and 20140206631A1; and to U.S. Pat. Nos. 9,662,363, 9,662,364, 9,662,365, 9,662,366, 9,669,062, 9,066,974, 9,095,606, 8,734,859, 8,597,695 and 8,541,382.

Each of these priority and related patents and patent applications is hereby incorporated by reference.

### BACKGROUND OF THE INVENTION

### 1. Field of the Invention

The present invention relates generally to wound healing, particularly to the use of combinations of certain herbs, certain herbal extracts and/or certain herbal molecular components, alone or in combination with known wound healing treatments, for enhancing migration of fibroblasts and/or keratinocytes to facilitate wound healing.

### 2. Description of the Related Art

Wound healing after injury is a complex process encompassing many cellular and biochemical events to restore tissue integrity. Wound healing generally involves re-epithelialization from the wound margin through keratinocyte proliferation and migration, and some growth factors are known to influence this process. See Syarina P. et al.; Wound healing potential of Spirulina platensis extracts on human dermal fibroblast cells. EXCLI Journal. 2015; 14:385-393, which is incorporated by reference. Human dermal fibroblasts also play significant roles in cutaneous wound repair and remodeling. They proliferate to expand, migrate into the wound bed, synthesize new ECM, and express thick actin bundles as myofibroblasts. It is desired to provide a treatment and/or supplement, including a safe and effective herbal combination, as a fibroblast and/or keratinocyte migration enhancer for improving skin wound healing efficiency.

### BRIEF DESCRIPTION OF THE DRAWINGS

FIGS. 1A-1B schematically illustrate example steps in a cooking process in accordance with certain embodiments.

FIG. 2 schematically illustrates example steps on a tablet formulation process in accordance with certain embodiments.

FIG. 3 illustrates the components of a shampoo in accordance with certain embodiments.

FIG. 4 illustrates a hair growth cycle and a hair fall control strategy in accordance with certain embodiments.

FIG. 5 illustrates certain targets of combinations of Rheum tanguticum, Rehmannia glutinosa and Lonicera japonica in accordance with certain embodiments.

FIGS. 6A-6E illustrate effects of different concentrations between 500:1 and 50:1 of Herb1 on migration of HFF-1 cells after 16 hours.

FIGS. 7A-7E illustrate effects of different concentrations between 500:1 and 50:1 of Herb2 on migration of HFF-1 cells after 16 hours.

FIGS. 8A-8E illustrate effects of different concentrations between 500:1 and 50:1 of Herb3 on migration of HFF-1 cells after 16 hours.

FIGS. 9A-9E illustrate effects of different concentrations between 500:1 and 50:1 of a combination of Herb 1, Herb 2 and Herb 3, at a ratio 1:1:3, on migration of HFF-1 cells after 16 hours.

FIG. 10A illustrates a baseline untreated wound at the start of a HFF-1 cell migration test.

FIG. 10B illustrates the baseline untreated wound of FIG. 10A after 16 hours.

FIG. 10C illustrates effects of the EGF baseline control solution on migration of HFF-1 cells after 16 hours.

FIGS. 11A-11E illustrate effects of different concentrations between 500:1 and 50:1 of Herb 1 on migration of HaCaT cells after 24 hours.

FIGS. 12A-12E illustrate effects of different concentrations between 500:1 and 50:1 of Herb2 on migration of HaCaT cells after 24 hours.

FIGS. 13A-13E illustrate effects of different concentrations between 500:1 and 50:1 of Herb3 on migration of HaCaT cells after 24 hours.

FIGS. 14A-14E illustrate effects of different concentrations, between 500:1 and 50:1, of a combination of Herb 1, Herb 2 and Herb 3, at a ratio 1:1:3, on migration of HaCaT cells after 24 hours.

FIG. 15A illustrates a baseline untreated wound at the start of a HaCaT cell migration test.

FIG. 15B illustrates the baseline untreated wound of FIG. 15A after 24 hours.

FIG. 15C illustrates effects of the EGF baseline control solution on migration of HaCaT cells after 24 hours.

### BRIEF DESCRIPTION OF THE TABLES

Table 1A shows a list of chemicals, reagents and details of media.

Table 1B shows a list of test items each along with the topically administered weight in grams used in the test.

Table 2 shows percentage cytotoxicities of test items in HFF-1 cells after 24 hours of treatment.

Table 3 shows quantitative wound healing effects of test items in HFF-lcells after 16 hours of treatment.

Table 4 shows percentage cytotoxicity of test items in HaCaT cells after 24 hours of treatment.

Table 5 shows quantitative wound healing effects of test items in HaCaT cells after 24 hours of treatment.

### DETAILED DESCRIPTION OF THE EMBODIMENTS

A method of enhancing skin wound healing efficiency is provided including administering to a patient with a skin wound a dosage composition that includes a fibroblast cell migration enhancing active component including at least 60 wt. % of an herbal combination consisting of between three and seven herbs at least including rheum tanguticum, lonicera japonica, and rehmannia glutinosa.

The method may include diluting the active component between 1:50 and 1:500 in an aqueous or other inactive solution configured for topical application directly on the skin wound.

In certain embodiments, the active component includes 30 mg or more of the herbal combination. For example, the active component may include 20.4 mg or more of the rehmannia glutinosa, 4 mg or more of the rheum tanguticum, and 5.6 mg or more of the lonicera japonica.

In certain embodiments, the active component includes 30 mg/ml or more of the herbal combination. For example, the active component may include 20.4 mg/ml or more of the rehmannia glutinosa, 4 mg/ml or more of the rheum tanguticum, and 5.6 mg/ml or more of the lonicera japonica. The dosage composition may include, in certain embodiments, between 60 ng/ml and 600 ng/ml of the herbal combination diluted in an aqueous or other inactive solution between 1:50 and 1:500.

The method may include repeating the administering of the dosage composition multiple times in a treatment regimen that includes administering over a duration of the treatment regimen 13.3 grams or more of the rehmannia glutinosa, 3.3 grams or more of the rheum tanguticum, and 3.3 grams or more of the lonicera japonica. Multiple doses of a topical formulation of the dosage composition may be administered at a skin wound location over the duration of the treatment regimen.

The active component may be combined with one or more inactive ingredients to produce a cream, lotion, ointment or other topical formulation configured for direct application at a location of said skin wound. The active component may be diluted within said topical formulation between 1:50 and 1:500. The active component may be combined with one or more inactive ingredients to produce a shampoo, conditioner, or other topical scalp or hair treatment.

An average particulate size of the active component may be reduced to less than 450 nm, 350 nm, 250 nm, 200 nm, 150 nm or 100 nm, or 50 nm. These nanoparticulates of the active component may be combined with one or more inactive ingredients to produce a nanogel formulation configured for direct application at a location of said skin wound.

The method may include formulating a combination therapy including administering a methotrexate regimen before, during, or after administration of the dosage composition, or combinations thereof.

A method of formulating a dosage composition for enhancing fibroblast cell migration and skin wound healing efficiency is also provided. The method includes formulating a fibroblast cell migration enhancing active component that includes at least 60 wt. % of an herbal combination consisting of between three and seven herbs at least including rheum tanguticum, lonicera japonica, and rehmannia glutinosa.

The method may include combining the active component with one or more inactive ingredients to produce a cream, lotion, ointment or other topical formulation configured for direct application at a location of a skin wound. The method may include diluting the active component within the topical formulation between 1:50 and 1:500. The method may include combining the active component with one or more inactive ingredients to produce a shampoo, conditioner, or other topical scalp or hair treatment.

An average particulate size of the active component may be reduced to less than 450 nm, 350 nm, 250 nm, 200 nm, 150 nm or 100 nm, or 50 nm. These nanoparticulates of the active component may be combined with one or more inactive ingredients to produce a nanogel formulation configured for direct application at a location of said skin wound.

The method may include formulating a combination therapy including administering a methotrexate regimen before, during, or after administration of the dosage composition, or combinations thereof.

A dosage composition for enhancing skin wound healing efficiency is also provided, including a fibroblast cell migration enhancing active component that includes at least 60 wt. % of an herbal combination consisting of between three and seven herbs at least including rheum tanguticum, lonicera japonica, and rehmannia glutinosa.

The active component may be diluted in an aqueous or other inactive solution between 1:50 and 1:500.

In certain embodiments, the active component of the dosage composition includes 30 mg or more of the herbal combination. For example, the active component may include 20.4 mg or more of the rehmannia glutinosa, 4 mg or more of the rheum tanguticum, and 5.6 mg or more of the lonicera japonica.

In certain embodiments, the active component includes 30 mg/ml or more of the herbal combination. For example, the active component may include 20.4 mg/ml or more of the rehmannia glutinosa, 4 mg/ml or more of the rheum tanguticum, and 5.6 mg or more of the lonicera japonica. The active component may include in certain embodiments between 60 ng/ml and 600 ng/ml of the herbal combination diluted in an aqueous or other inactive solution between 1:50 and 1:500.

The dosage composition may be formulated as a cream, lotion, ointment or other topical formulation configured for direct application at a location of a skin wound. The active component may be diluted within the topical formulation between 1:50 and 1:500.

The dosage composition may be formulated as a shampoo, conditioner, or other topical scalp or hair treatment.

An average particulate size of the active component may be reduced to less than 450 nm, 350 nm, 250 nm, 200 nm, 150 nm or 100 nm, or 50 nm. These nanoparticulates of the active component may be combined with one or more inactive ingredients to produce a nanogel formulation configured for direct application at a location of said skin wound.

A combination therapy may include a methotrexate regimen configured for administration before, during, or after administration of the dosage composition, or combinations thereof.

A treatment regimen is also provided that includes multiple doses of a composition for enhancing fibroblast cell migration and skin wound healing efficiency, including a housing containing multiple doses that each include a fibroblast cell migration enhancing active component including at least 60 wt. % of an herbal combination consisting of between three and seven herbs at least including rheum tanguticum, lonicera japonica, and rehmannia glutinosa. In certain embodiments, the multiple doses may each contain 30 mg or more of the herbal combination, and the housing may contain 13.3 grams or more of the rehmannia glutinosa, 3.3 grams or more of the rheum tanguticum, and 3.3 grams or more of the lonicera japonica.

The active component of the treatment regimen may be combined with one or more inactive components to produce a cream, lotion, ointment or other topical formulation, that may be periodically or otherwise directly applied multiple times at a skin wound location over a duration of the treatment regimen, or alternatively as a tablet, gel cap or other oral formulation, or as a subcutaneous or IV formulation. The active component may be diluted in an aqueous or other inactive solution within a topical or other formulation between 1:50 and 1:500.

The active component of the treatment regimen may be combined with one or more inactive components for producing a shampoo, conditioner, or other topical scalp or hair treatment.

In certain example embodiments, a dosage composition may include an effective dose between 20-160 mg/kg of an herbal combination of Rheum tanguticum, Rehmannia glutinosa and Lonicera japonica that may be administered to a patient by topical application at a skin wound location, or alternatively orally, subcutaneously or intravenously. A dosage composition in accordance with certain example embodiments may include an effective dose between 1.0 wt. %-15 wt. % of an herbal combination of Rheum tanguticum, Rehmannia glutinosa and Lonicera japonica that may be administered to a patient by topical application at a skin wound location, or alternatively orally, subcutaneously or intravenously. Effective doses of the medicine may include 1.0 wt. %-15.0 wt. % of said herbal combination of Rheum tanguticum, Rehmannia glutinosa and Lonicera japonica. Effective doses may include 2.5 wt. %-5.0 wt. % of the herbal combination of Rheum tanguticum, Rehmannia glutinosa and Lonicera japonica in certain embodiments depending on an extent of one or more skin wounds on a patient.

A dosage composition in certain example embodiments may further include, or be administered simultaneously or at staggered intervals relative to administration of one or more additional medicines that may be effective in countering or treating dermal or epidermal inflammation, neutrophil, monocyte chemotaxis, antimicrobial defense mechanisms, pruritus, acanthosis, parakeratosis, hyperkeratosis, and/or angiogenesis.

The dosage composition may be formulated in various ways including as a shampoo, conditioner, cream, lotion, ointment or other topical skin, scalp or hair treatment or a pill, skin patch, injection pen, subdermal injection, IV fluid, tablet, capsule, lipid carrier, nano-crystal or other nano-particulate formulation, subcutaneous insert, or stent, or combinations thereof.

Effective doses or regimens of multiple doses may include up to 1-6 grams of said herbal combination for a 50 kg patient or up to 2-12 grams for a 100 kg patient. Depending on the extent of the skin wound, more or less of said herbal combination may be applied along an extent of one or more skin wound locations of a patient.

Effective doses or regimens of multiple doses may include 0.2-1.2 grams of Rheum tanguticum, 0.6-3.6 grams of Rehmannia glutinosa and 0.2-1.2 grams of Lonicera japonica for some patients and particularly for an approximately 50 kg patient or 0.4-2.4 grams of Rheum tanguticum, 1.2-7.2 grams of Rehmannia glutinosa and 0.4-2.4 grams of Lonicera japonica for an approximately 100 kg patient.

### Example Dosage Compositions and Combination Therapies

Effective doses of an herbal combination of Rheum tanguticum, Rehmannia glutinosa and Lonicera japonica have been demonstrated in mice between 1500 mg/kg and 500 mg/kg. Very low doses of 250 mg/kg may also be administered with modest effectiveness and elevated doses between 1500 mg/kg and 3000 mg/kg may be administered as tolerated with enhanced curative potential. This effective dose range in humans is approximately between 162.0 mg/kg and 40.50 mg/kg, while very low formulations of 20.25 mg/kg and high doses between 162 mg/kg and 324 mg/kg are also capable of formulation. The proportions of the three herbs have been shown to be effective at approximately one part Rheum tanguticum, one part Lonicera japonica and between two and four parts Rehmannia glutinosa.

An elevated dose or regimen of multiple doses example for a 100 kg patient includes 2.4 grams Rheum tanguticum, 2.4 grams Lonicera japonica and 7.2 grams Rehmannia glutinosa. For a 50 kg patient, an elevated dose example includes 1.2 grams Rheum tanguticum, 1.2 grams Lonicera japonica and 3.6 grams Rehmannia glutinosa.

A medium dose or regimen of multiple doses example for a 100 kg patient includes 1.6 grams Rheum tanguticum, 1.6 grams Lonicera japonica and 4.8 grams Rehmannia glutinosa. For a 50 kg patient, a medium dose example includes 0.8 grams Rheum tanguticum, 0.8 grams Lonicera japonica and 2.4 grams Rehmannia glutinosa.

A low dose or regimen of multiple doses example for a 100 kg patient includes 0.8 grams Rheum tanguticum, 0.8 grams Lonicera japonica and 2.4 grams Rehmannia glutinosa. For a 50 kg patient, a medium dose example includes 0.4 grams Rheum tanguticum, 0.4 grams Lonicera japonica and 1.2 grams Rehmannia glutinosa.

A very low dose or regimen of multiple doses example for a 100 kg patient includes 0.4 grams Rheum tanguticum, 0.4 grams Lonicera japonica and 1.2 grams Rehmannia glutinosa. For a 50 kg patient, a medium dose example includes 0.2 grams Rheum tanguticum, 0.2 grams Lonicera japonica and 0.6 grams Rehmannia glutinosa.

An elevated dose or regimen of multiple doses example for a 100 kg patient includes 2.4-4.8 grams Rheum tanguticum, 2.4-4.8 grams Lonicera japonica and 7.2-14.4 grams Rehmannia glutinosa. For a 50 kg patient, a high dose example includes 1.2-2.4 grams Rheum tanguticum, 1.2-2.4 grams Lonicera japonica and 3.6-7.2 grams Rehmannia glutinosa.

In accordance with these examples, formulations may be prepared for and administered to 100 kg patients that include 0.4-4.8 grams Rheum tanguticum, 0.4-4.8 grams Lonicera japonica and 1.2-14.4 grams Rehmannia glutinosa, and to 50 kg patients including 0.2-2.4 grams Rheum tanguticum, 0.2-2.4 grams Lonicera japonica and 0.6-7.2 grams Rehmannia glutinosa. A moderate range example for 100 kg patients includes 0.8-2.4 grams Rheum tanguticum, 0.8-2.4 grams Lonicera japonica and 2.4-7.2 grams Rehmannia glutinosa, and that for 50 kg patients includes 0.4-1.2 grams Rheum tanguticum, 0.4-1.2 grams Lonicera japonica and 1.2-3.6 grams Rehmannia glutinosa. These example dosage compositions may be administered multiple times in a treatment regimen lasting a few days or weeks or even months at intervals of a few hours to daily, every other day or as needed.

In another example, a treatment regimen may include 1-10 gram daily doses of combinations of Rheum tanguticum, Rehmannia glutinosa and Lonicera japonica. A treatment regimen may include 2-5 gram daily doses, or approximately 3 gram daily doses. In the 3.0 gram daily dose example, 1.5 gram doses may be administered twice daily, i.e., 1.5 grams twice a day. A total daily dose may be administered in the form of four (4) tablets, e.g., 2 tablets, twice a day, of 1.0 gram each containing 750 mg of a combination of Rheum tanguticum, Rehmannia glutinosa and Lonicera japonica as well as 250 mg of excipients.

### Example Formulations

In addition to the above active herbal combination ingredient, a medicinal tablet composition is provided for example for oral administration that may include one or more of the following further ingredients: 125 mg of MCC (Avicel), 20 mg of talc powder, 30 mg of aerosol 200, 10 mg of croscarmellose sodium and/or 30 mg of PVPK 30. This example may also include one or more of the following lubrication ingredients: 20 mg of croscarmellose sodium, 5 mg of aerosol 200, 20 mg of talc powder and/or 10 mg of magnesium sterate.

A medicinal cream is also provided for topical administration that includes the above active herbal combination ingredient, in an example concentration of 2.5%, and one or more of the following further ingredients: 2% sesame oil or sesamum indicum (sesame) seed oil, 4% cetostearyl alcohol or cetearyl alcohol, 5% arlacel 165 or glyceryl stearate or PEG-100 stearate, 3% light liquid paraffin or mineral oil, 2% cresmer wax EW or ceteareth-20 or cetearyl alcohol, 3% stearyl stearate, 0.2% butyl hydroxyl toluene or BHT, 4.2% propylene glycol, 0.2%potassium sorbate, 0.2% sodium benzoate, 5% glycerol or glycerin, 0.1% sandal wood oil or santalum album (sandalwood) oil, and 68.6% water.

A medicinal cream is also provided for topical administration that includes the above active herbal combination ingredient, in aqueous extract and in an example concentration of 2.5%, and one or more of the following further ingredients: till oil, cetostearyl alcohol, arlacel 165, light liquid paraffin, cresmer wax EW, stearyl stearate, butyl hydroxyl toluene, propylene glycol, sodium methyl paraben, sodium propyl paraben, glycerol, sandalwood oil. A cream placebo may be formulated for clinical trials that substitutes F-24 chocolate brown color TAS and brilliant blue color for the above active herbal combination ingredient.

### Preparation of Topical, Oral or Subdermal Medicine

Treatments described herein may be prepared for topical use for enhancing migration of fibroblasts and improving skin wound healing efficiency, and/or for treating melanoma, eczema, dermatisis, BCC (basal cyr carcinoma) and/or inflammatory skin diseases like Psoriasis. For example, combinations of herbs and/or herbal extracts as described herein may be prepared as a cream to apply onto the skin. Another known or discovered treatment may be included or may be administered separately before, during or after the herbal treatment. The other known or discovered treatment, along with herbal combinations and/or herbal extract combinations described herein may be injected to infected areas of the skin of a patient using a syringe. An example method for preparation of an external cream in accordance with certain embodiments is provided below.

First, two of more of the herbs may be cooked, for example, as described elsewhere herein or as may be understood or determined by those skilled in the art. A cream is then prepared that may be somewhat more of less than half herbs and half cream, e.g., a 25%-75% liquid of herbs in 1:1 ratio and 25%-75% cream may be used. The herbs can also be prepared as a tincture, e.g., soaking the herbs in alcohol for a period of time such as 2 weeks in a ratio of 1:3, for example. This herbal liquid can then be mixed with the cream in the same way as described above.

The herbs may be prepared for cooking by grinding and/or homogenizing the herbs. Grinding may be achieved using a Dyno-mill run once to obtain, e.g., 250 nm particle sizes or multiple times down to, e.g., 150 nm particle sizes. A high pressure homogenizer may be used, whereby the mixture is pushed through a filter, e.g., a 0.2 μm filter. Sonication or ultrasound may also be used.

Straining may be performed to get an aqueous extract. Lyophilization or freeze drying may be performed to prepare the mixture for in vitro use. Solubilization may be performed along with selection of a concentration of the extract. A cream, tablet, capsule, nano-lipid carrier, nanogel, or nano-chrystals, or nano-particulates may be formulated for administration to human or animal patients.

Treatments described herein may also be effective against immunodeficiency diseases such as HIV and AIDS, as well as other conditions affecting or caused by disorders of the immune system. Herbal combinations of one or more of Rheum tanguticum, Rehmannia glutinosa and Lonicera japonica and/or one or more other herbs or molecules described herein may be administered as a nutritional supplement or as a supplement to an exercise regimen or as an energy supplement or as a pain relief supplement or as a diuretic or sleep aid. NSAIDs, such as ibuprofen, naproxen and aspirin, other non-steroidal anti-inflammatories, acetaminophen, and/or steroidal anti-inflammatories may be combined with an herbal combination of one or more of Rheum tanguticum, Rehmannia glutinosa and Lonicera japonica and/or one or more other herbs or molecules described herein, with or without administration of a known or discovered treatment, before, during or after the herbal treatment, to treat inflammation or other ailments that are commonly treatable with NSAIDs, including chronic pain. Formulations may be prepared for oral or topical administration as long release, lipidized dosage compositions or as short release non-lipidized formulas.

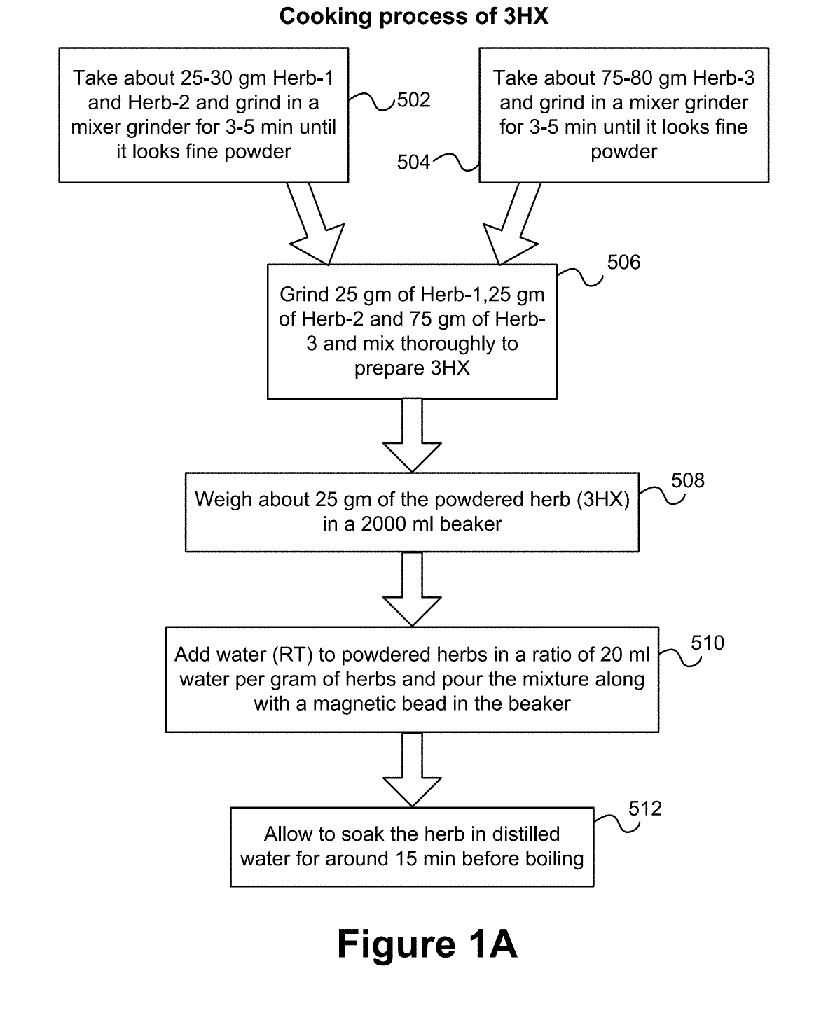

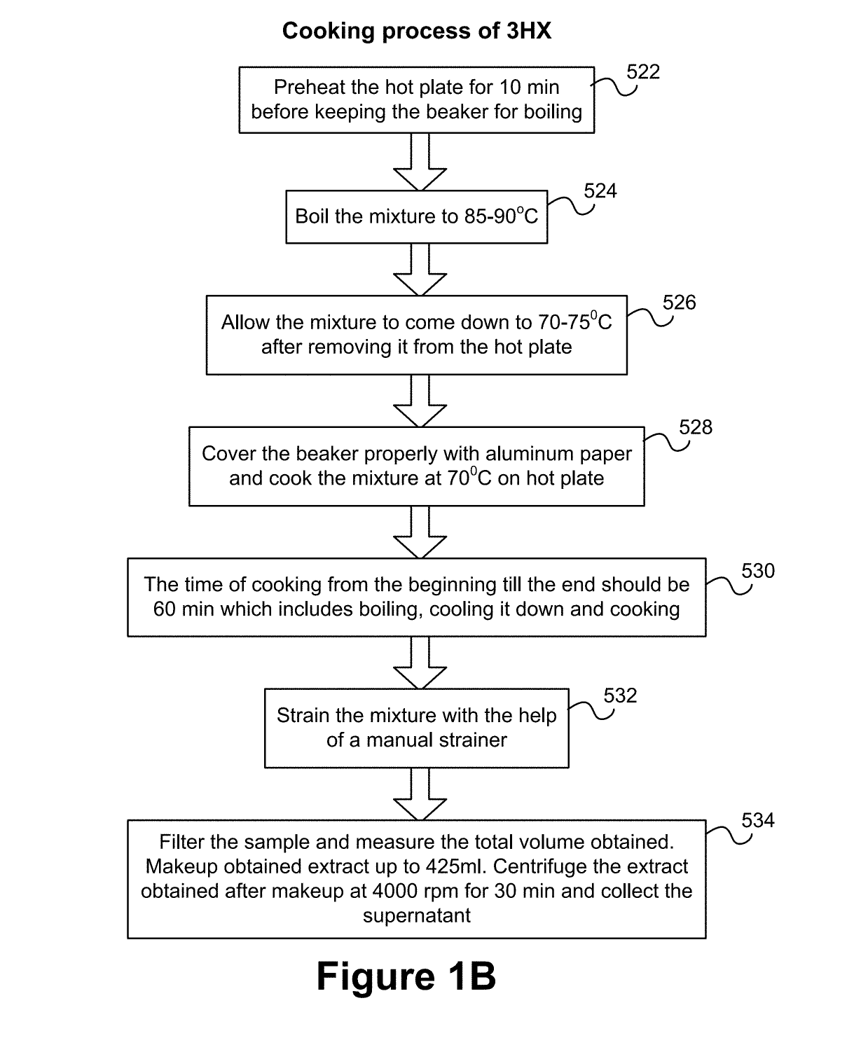

A cooking process may be performed as in the example of FIGS. 1A-1B. Referring to FIG. 1A, a step 502 in a cooking process may include taking about 25-30 grams of a first herb, e.g., rheum tanguticum, and a second herb, e.g., Lonicera japonica, and grinding in a mixer grinder for three to five minutes or until a fine powder has been mixed and ground. A step 504 in the process may include taking about 75-80 grams of a third herb, e.g., Rehmannia glutinosa, and grinding in a mixer grinder for three to five minutes or until a fine powder has been mixed and ground.

A step 506 in the cooking process may further include grinding 25 grams of the first herb, e.g., rheum tanguticum, 25 grams of the second herb, e.g., Lonicera japonica, and 75 grams of the third herb, e.g., Rehmannia glutinosa, and mixing thoroughly to prepare a three herb combination (“3HX”).

A next step 508 may include weighing about 25 grams of the powdered three herb combination 3HX in a 2000 ml beaker or other suitable container. A next step 510 may include adding water (RT) to the 25 grams of powdered three herb combination 3HX in a ratio of about 20 ml water per gram of powdered three herb combination and pouring the mixture along with a magnetic bead into the beaker. A next step 512 may include allowing the aqueous three herb combination to soak in distilled water for around 15 minutes before boiling.

Referring now to FIG. 1B, a hot plate may be pre-heated, for 10 minutes, e.g., before keeping the beaker for boiling, as indicated at step 522. A step 524 may then include boiling the mixture to 85-90° C. A step 526 may include allowing the mixture to come down to 70-75° C. after removing the mixture from the hot plate. A step 528 may include covering the beaker properly with aluminum paper and cooking the mixture at 70° C. on a hot plate. A time of cooking in accordance with the example of step 530 from the beginning to the end should be around 60 minutes which includes boiling, cooling and cooking.

A next step 532 may include straining the mixture with the help of a manual strainer. A step 534 may involve filtering the sample and measuring the total volume obtained, as well as making up an obtained extract up to 425 ml, centrifuging the extract obtained after making up at 4000 rpm for 30 minutes and collecting the supernatant.

A cream or lotion or shampoo or ointment or patch or other topical formulation may be prepared. Also, a pill or container of pills may be prepared in lipidized or non-lipidized form, and coated or uncoated for extended release, timed release, sustained release, modified release, immediate release, quick release, delayed release, controlled release, controlled delivery, long-acting or sustained action. Also, an IV or subdermal injection fluid may be formulated.

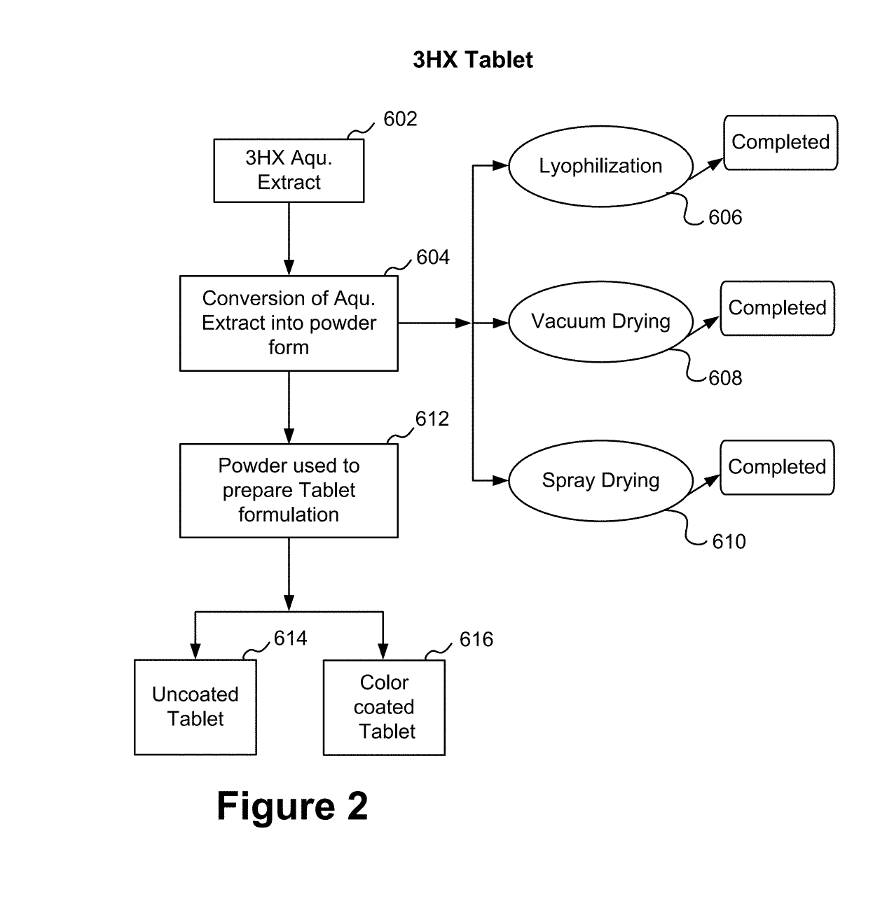

In the example of FIG. 2, an aqueous extract, e.g., including a herein-described one, two or three (or more) herb combination is prepared, provided or acquired at step 602. A step 604 may include converting the aqueous extract into powder form. The converting into powder form of step 604 may include one or more of lyophilization or freeze drying 606, vacuum drying 608 and/or spray drying 610. The drying may include heating or proximate hygroscopic disposition or spinning or otherwise as may understood by those skilled in the art.

At step 612, the lyophilized or otherwise dried one, two or three (or more) herb powder may be used to formulate a tablet, pill, capsule or other orally-administered formulation. Such orally-administered formulation may be uncoated 614 or color coated 616. Batch sizes may be in multiples of one or more thousand.

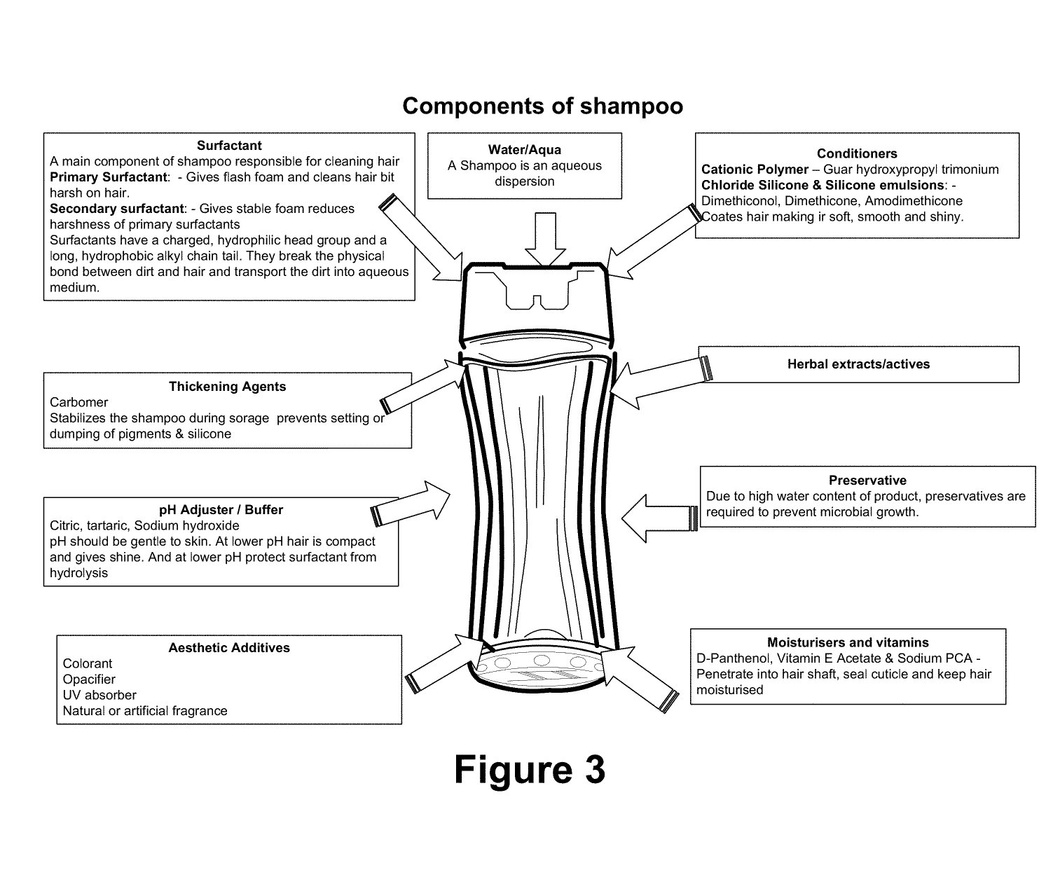

Referring to FIG. 3, an example of a shampoo in accordance with certain embodiments is illustrated as including nine generalized components. These nine components include a surfactant, a thickening agent, a pH adjuster/buffer, an aesthetic additive, water, a conditioner, one or more active herbs or herbal extracts or molecules, a preservative and moisturizers/vitamins. Shampoos in accordance with various alternative formulations may include fewer than all of these nine components and they may include other active or inactive components known to those skilled in the art as having some advantage when included in a shampoo formula or in a medicinal combination for treating a skin condition such as psoriasis, eczema, melanoma, or dermatitis or hair loss or another head or scalp disorder or ailment.

A shampoo in accordance with certain embodiments includes one or more surfactants that may be known or discovered as being advantageous for cleaning hair with a shampoo. A primary surfactant may be included to provide flash foam for cleaning the hair by removing dirt and other impurities. A secondary surfactant may be included to provide stable foam and to reduce the harshness of the primary surfactant. A surfactant may be used that includes a charged, hydrophilic head group and a long, hydrophobic alkyl chain tail. Surfactants are configured to break molecular bonds between dirt and hair and to transport the dirt into an aqueous medium to be rinsed free from the hair and scalp. Examples of surfactants that may be contained in a shampoo in accordance with certain embodiments include sodium laureth sulphate, ammonium laureth sulfate, and sodium cocoyl isethionate. Examples of co-surfactants include cocamide MEA and cocoamidopropyl betaine.

A shampoo in accordance with certain embodiments may include a thickening or suspending agent. Examples of thickening or suspending agents that may be contained in accordance with certain embodiments include carbomer and PEG 150 distearate. The thickening agent may be included to stabilize the shampoo during storage and/or to prevent the setting or dumping of pigments and silicone.

A pH adjuster or buffer may be included in a shampoo in accordance with certain embodiments. An example of a pH adjuster or buffer includes citric acid, tartaric and sodium hydroxide. The pH adjuster or buffer is configured to cause the shampoo to be gentle to the skin. A lower pH may cause hair to be compact and to shine and to protect the surfactant from hydrolysis, and as such, the pH adjuster or buffer may serve to lower the pH of the shampoo. However, alternative embodiments include pH adjusters that serve to raise the pH of a shampoo that contains an herbal formula that exhibits an exceptionally low pH.

An aesthetic additive may be included in a shampoo in accordance with certain embodiments. Examples of aesthetic additives include colorants, opacifiers, UV absorbers, perfumes and natural and artificial fragrances.

One or more conditioners may be included in a shampoo in accordance with certain embodiments. The one or more conditioners may include a cationic polymer such as guar hydroxypropyl trimonium chloride. The one or more conditions may include silicone and/or a silicone emulsion such as dimethiconol, dimethicone, or amodimethicone. The silicone and/or silicone emulsion may serve to coat the hair and cause the hair to become soft, smooth and shiny.

A shampoo in accordance with certain embodiments includes one or more active herbs or herbal extracts or emotives that are described in several examples herein. These one or more active herbs or herbal extracts serve to promote treatment of certain hair and scalp conditions such as psoriasis, eczema, dermatitis, melanoma, hair loss and other hair or scalp conditions described herein or understood by those skilled in the art.

A preservative may be included in a shampoo in accordance with certain embodiments. The preservative may be configured to prevent microbial growth. Examples of preservatives that may be contained in a shampoo in accordance with certain embodiments include paraben free, formaldehyde donor free and halogenated free.

A moisturizer and/or one or more vitamins may be included in a shampoo in accordance with certain embodiments. Examples include combinations of D-Panthenol, vitamin E acetate, sodium PCA, glycerine and one or more amino acids. The moisturizer and/or vitamins may be configured to penetrate into hair shaft, seal cuticles and keep hair moisturized.

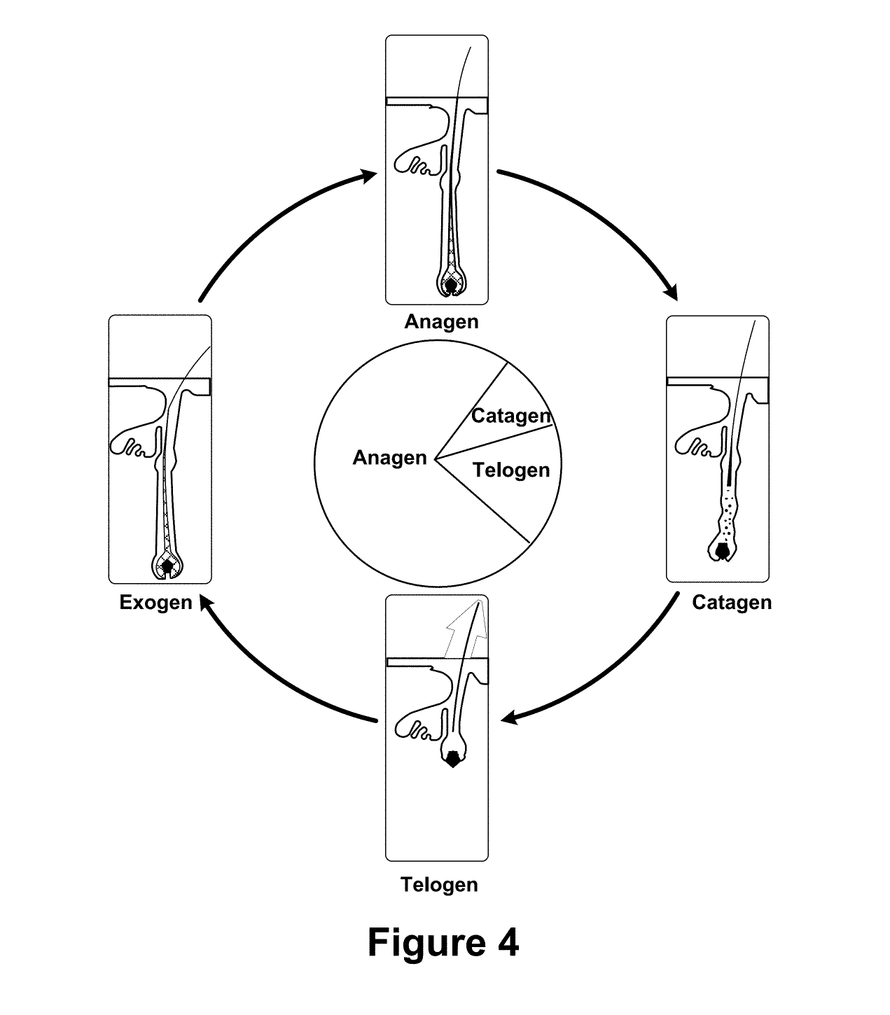

A shampoo in accordance with certain embodiments may include a hydro-alcoholic hair serum. Referring to FIG. 4, a hair growth cycle includes exogen, anagen, catagen and tetogen phases. The anagen phase involves active hair growth, whereby hair follicles regenerate and generate pigmented hair shafts. The telogen phase is a resting phase. The catagen phase involves cessation of hair growth and pigmentation, and release of papilla from the bulb. A hydro-alcoholic hair serum may be configured as a concentrated product that is typically left on the hair for a more extended duration than an ordinary shampoo with a typical shower routine. The hydro-alcoholic hair serum may be configured to be light and non-sticky on the scalp and as a non-irritant, to be light and non-sticky on the hair, to have little or no effect on hair volume, to strengthen scalp and DPC, to provide keratinization and collagen synthesis, to promote hair growth or to control hair fall, or combinations thereof. For example, the attributes of a hydro-alcoholic hair serum in accordance with certain embodiments may assist or promote treatment of psoriasis, seborrhea dandruff or hair fall. A hydro-alcoholic hair serum in accordance with certain embodiments may include water, alcohol, humectant, solubilizer, water-based polymer, scalp conditioner, niacinamide, caffeine and panthenol. The ratio of alcohol, water and solubilizer may be adjusted depending of the solubilization power of the active herbal treatment composition.

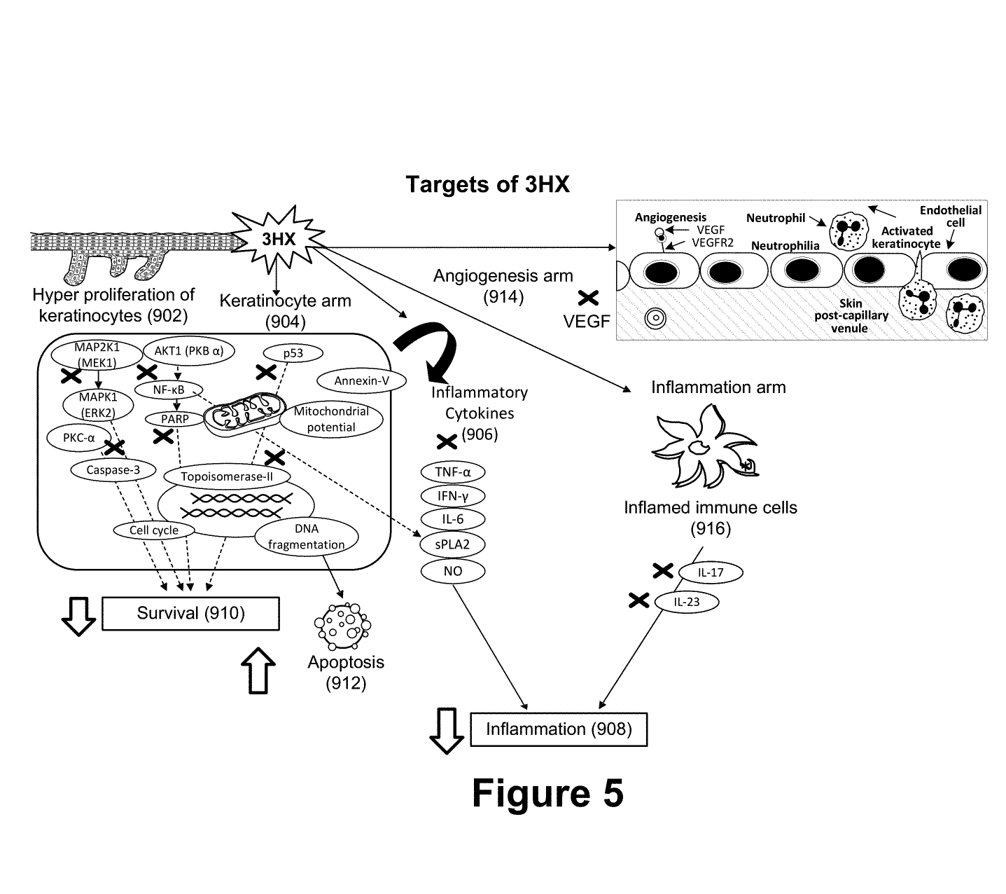

FIG. 5 schematically illustrates certain targets of medicinal compositions including combinations of Rheum tanguticum, Rehmannia glutinosa and Lonicera japonica (3HX), in accordance with certain embodiments. FIG. 5 illustrates a hyper proliferation of keratinocytes 902, e.g., as may be present in a patient suffering with an inflammatory skin condition, or melanoma, or an autoimmune or other disorder involving the skin, or combinations thereof. 3HX may be administered to the patient to target keratinocyte-related activities 904, e.g., to inhibit MAP2K1 (MEK1) and MAPK1 (ERK2), AKT1 (PKB alpha) and NF-kB, p53, PKC alpha, PARP, topoisomerase-II, e.g., influencing Annexin-V, mitochondrial potential leading to inflammatory cytokines 906 and inflammation 908, Caspase-3 and/or cell cycles toward keratinocyte survival 910 and/or DNA fragmentation toward apoptosis 912.

FIG. 5 also illustrates that 3HX may be administered to a patient to target inflammatory cytokine activity 906 such as that of TNF alpha, IFN gamma, IL-6, sPLA2 and/or NO that may otherwise lead to inflammation 908.

FIG. 5 also illustrates that 3HX may be administered to a patient to target angiogenesis activity 914 such as that of VEGF, VEGFR2 and/or neutrophil that may activate keratinocytes and influence endothelial cells and/or skin post-capillary venules.

Any of Rheum tanguticum, Rehmannia glutinosa and Lonicera japonica may be administered as a single herb complement or supplement along with another known or discovered treatment regimen. Any two of these herbs, or all three of them, may also be combined and administered as a supplement or complement to another known or discovered treatment regimen. In certain embodiments, the regimen described includes 3.3 grams to 60 grams of Lonicera japonica. In other embodiments, one or more molecules, molecular extracts and/or molecular compounds are included in the regimen. In further embodiments, one, two, three or all four of the herbs Mu Dan Pi, Xian He Cao, Chun Gen Pi and Di Gu Pi is/are combined with one, two or all of the three herbs Rehmannia glutinosa, Rheum tanguticum and Lonicera japonica, including 3.3 grams to 15 grams of any of the other four of the seven herbs, e.g., 3.3 grams to 15 grams of Mu Dan Pi, 3.3-10 grams to 15 grams of Xian He Cao, 3.3-10 grams to 15 grams of Chun Gen Pi, and/or 3.3-5 grams to 15 grams of Di Gu Pi.

In another example, a known or discovered treatment regimen is supplemented with a low dose formula including the three herbs including 3.3-20 grams of Rheum tanguticum, 10-60 grams of Rehmannia glutinosa, and 3.3-20 grams of Lonicera japonica.

In another example, a known or discovered treatment regimen is supplemented with a low dose formula including 3.3-20 grams of Rheum tanguticum and 10-60 grams of Rehmannia glutinosa.

In another example, a known or discovered treatment regimen is supplemented with a low dose formula including 3.3-20 grams of Lonicera japonica and 10-60 grams of Rehmannia glutinosa.

In another example, a known or discovered treatment regimen is supplemented with a low dose formula including 3.3-20 grams of Rheum tanguticum and 3.3-20 grams of Lonicera japonica.

In another example, a known or discovered treatment regimen is supplemented with a low dose formula including the one herb including 3.3-30 grams of Rheum tanguticum.

In another example, a known or discovered treatment regimen is supplemented with a low dose formula including the one herb including 3.3-30 grams of Lonicera japonica.

In another example, a known or discovered treatment regimen is supplemented with a low dose formula including the one herb including 10-90 grams of Rehmannia glutinosa. In another example, a known or discovered treatment regimen is supplemented with a medium dose formula including the three herbs including 10-100 grams of Rheum tanguticum, 25-250 grams of Rehmannia glutinosa, and 10-100 grams of Lonicera japonica.

In another example, a known or discovered treatment regimen is supplemented with a medium dose formula including 10-100 grams of Rheum tanguticum and 25-250 grams of Rehmannia glutinosa.

In another example, a known or discovered treatment regimen is supplemented with a medium dose formula including 10-100 grams of Lonicera japonica and 25-250 grams of Rehmannia glutinosa.

In another example, a known or discovered treatment regimen is supplemented with a medium dose formula including 10-100 grams of Rheum tanguticum and 10-100 grams of Lonicera japonica.

In another example, a known or discovered treatment regimen is supplemented with a medium dose formula including the one herb including 10-100 grams of Rheum tanguticum.

In another example, a known or discovered treatment regimen is supplemented with a medium dose formula including the one herb including 10-100 grams of Lonicera japonica.

In another example, a known or discovered treatment regimen is supplemented with a medium dose formula including the one herb including 25-250 grams of Rehmannia glutinosa.

In another example, a known or discovered treatment regimen is supplemented with a high dose formula that includes 30-300 grams of Rheum tanguticum, 60-600 grams of Rehmannia glutinosa, and 30-300 grams of Lonicera japonica.

In another example, a known or discovered treatment regimen is supplemented with a high dose formula that includes 30-300 grams of Rheum tanguticum, and 60-600 grams of Rehmannia glutinosa.

In another example, a known or discovered treatment regimen is supplemented with a high dose formula that includes 30-300 grams of Rheum tanguticum and 30-300 grams of Lonicera japonica.

In another example, a known or discovered treatment regimen is supplemented with a high dose formula that includes 60-600 grams of Rehmannia glutinosa and 30-300 grams of Lonicera japonica.

In another example, a known or discovered treatment regimen is supplemented with a high dose formula that includes 30-300 grams of Rheum tanguticum.

In another example, a known or discovered treatment regimen is supplemented with a high dose formula that includes 30-300 grams of Lonicera japonica.

In another example, a known or discovered treatment regimen is supplemented with a high dose formula that includes 60-600 grams of Rehmannia glutinosa.

In another example, a known or discovered treatment regiment is supplemented with an herbal combination that includes 25-75 grams of Rheum tanguticum, 45-135 grams of Rehmannia glutinosa, and 25-75 grams of Lonicera japonica in about 250-750 grams of water, or in dry or lipidized form for oral, topical, sub-dermal or IV administration.

In another example, a known or discovered treatment regimen is supplemented with a low dose herbal formula that includes 3.3-10 grams of Rheum tanguticum, and 13.3-40 grams of Rehmannia glutinosa in about 100-300 grams of water, or in dry or lipidized form for oral, topical, sub-dermal or IV administration.

In another example, a known or discovered treatment regimen is supplemented with a high dose herbal formula that includes 30-100 grams of Rheum tanguticum and 60-200 grams of Rehmannia glutinosa in about 600-6000 grams of water, or in dry or lipidized form for oral, topical, sub-dermal or IV administration.

In another example, a known or discovered treatment regimen is supplemented with a medium dose herbal formula that includes 10-50 grams of Rheum tanguticum and 30-150 grams of Rehmannia glutinosa in about 400-2000 grams of water, or in dry or lipidized form for oral, topical, sub-dermal or IV administration.

In another example, a known or discovered treatment regimen is supplemented with a medium dose herbal formula that includes 20-50 grams of Rheum tanguticum and 40-100 grams of Rehmannia glutinosa in about 600-5000 grams of water, or in dry or lipidized form for oral, topical, sub-dermal or IV administration.

In another example, a known or discovered treatment regimen is supplemented with a medium dose herbal formula that includes 15-100 grams of Rheum tanguticum and 45-300 grams of Rehmannia glutinosa in about 600-4000 grams of water, or in dry or lipidized form for oral, topical, sub-dermal or IV administration.

In another example, a known or discovered treatment regimen is supplemented with a medium dose herbal formula that includes 25-75 grams of Rheum tanguticum in about 300 grams water, and 50-150 grams of Rehmannia glutinosa in about 500 grams of water, or in dry or lipidized form for oral, topical, sub-dermal or IV administration.

### Low Dose Formulations and Supplements

In an example involving a combination of a known or discovered regimen and a low dose combination of the three herbs, 0.01-0.4 grams of Rehmannia glutinosa in 1-6 grams of water, 0.01-0.2 grams of Rheum tanguticum in 0.1-6 grams of water and 0.01-0.4 grams of Lonicera japonica in 0.1-6 grams of water may be combined in a single or multiple treatment dosage regimen and administered to a patent. The combination may be cooked once or twice, and the herb:liquid ratio may be 1:10 or otherwise, and the herbs may be prepared in multiple ways, including as dry, aqueous or lipidized components. The doses may be administered every 1-10 days or multiple times daily including 2-6 times daily, and even as often as 10 times daily.

In another example, a known or discovered treatment regimen is supplemented with a low dose formula including the three herbs including 0.3-3.3 grams of Rheum tanguticum , 1-6 grams of Rehmannia glutinosa, and 0.3-3.3 grams of Lonicera japonica.

In another example, a known or discovered treatment regimen is supplemented with a low dose formula including 0.3-3.3 grams of Rheum tanguticum and 1-6 grams of Rehmannia glutinosa.

In another example, a known or discovered treatment regimen is supplemented with a low dose formula including 0.3-3.3 grams of Lonicera japonica and 1-6 grams of Rehmannia glutinosa.

In another example, a known or discovered treatment regimen is supplemented with a low dose formula including 0.3-3.3 grams of Rheum tanguticum and 0.3-3.3 grams of Lonicera japonica.

In another example, a known or discovered treatment regimen is supplemented with a low dose formula including the one herb including 0.3-3.3 grams of Rheum tanguticum.

In another example, a known or discovered treatment regimen is supplemented with a low dose formula including the one herb including 0.3-3.3 grams of Lonicera japonica.

In another example, a known or discovered treatment regimen is supplemented with a low dose formula including the one herb including 0.1-10 grams of Rehmannia glutinosa.

In another example, a known or discovered treatment regimen is supplemented with a low dose formula including the three herbs including 0.1-10 grams of Rheum tanguticum, 0.25-25 grams of Rehmannia glutinosa, and 0.10-10 grams of Lonicera japonica.

In another example, a known or discovered treatment regimen is supplemented with a low dose formula including 0.10-10 grams of Rheum tanguticum and 0.25-25 grams of Rehmannia glutinosa.

In another example, a known or discovered treatment regimen is supplemented with a low dose formula including 0.1-10 grams of Lonicera japonica and 0.25-25 grams of Rehmannia glutinosa.

In another example, a known or discovered treatment regimen is supplemented with a low dose formula including 0.1-10 grams of Rheum tanguticum and 0.1-10 grams of Lonicera japonica.

In another example, a known or discovered treatment regimen is supplemented with a low dose formula including the one herb including 0.1-10 grams of Rheum tanguticum.

In another example, a known or discovered treatment regimen is supplemented with a low dose formula including the one herb including 0.1-10 grams of Lonicera japonica.

In another example, a known or discovered treatment regimen is supplemented with a low dose formula including the one herb including 0.25-25 grams of Rehmannia glutinosa.

In another example, a known or discovered treatment regimen is supplemented with a low dose formula that includes 0.3-3 grams of Rheum tanguticum, 0.6-6 grams of Rehmannia glutinosa, and 0.3-3 grams of Lonicera japonica.

In another example, a known or discovered treatment regimen is supplemented with a low dose formula that includes 0.3-3 grams of Rheum tanguticum, and 0.6-6 grams of Rehmannia glutinosa.

In another example, a known or discovered treatment regimen is supplemented with a low dose formula that includes 0.3-3 grams of Rheum tanguticum and 0.3-3 grams of Lonicera japonica.

In another example, a known or discovered treatment regimen is supplemented with a low dose formula that includes 0.6-6 grams of Rehmannia glutinosa and 0.3-3 grams of Lonicera japonica.

In another example, a known or discovered treatment regimen is supplemented with a low dose formula that includes 0.3-3 grams of Rheum tanguticum.

In another example, a known or discovered treatment regimen is supplemented with a low dose formula that includes 0.3-3 grams of Lonicera japonica.

In another example, a known or discovered treatment regimen is supplemented with a low dose formula that includes 0.6-6 grams of Rehmannia glutinosa.

In another example, a known or discovered treatment regiment is supplemented with a low dose herbal formula that includes 0.1-10 grams of Rheum tanguticum in about 1-100 grams water, or in dry or lipidized form for oral, topical, sub-dermal or IV administration.

In another example, a known or discovered treatment regiment is supplemented with a low dose herbal formula that includes 0.25-25 grams of Rehmannia glutinosa in about 2.5-250 grams of water, or in dry or lipidized form for oral, topical, sub-dermal or IV administration.

In another example, a known or discovered treatment regimen is supplemented with a low dose herbal formula that includes 0.1-10 grams of Lonicera japonica in about 1.0-100 grams of water, or in dry or lipidized form for oral, topical, sub-dermal or IV administration.

In another example, a known or discovered treatment regimen is supplemented with a low dose herbal formula that includes 0.2-2.0 grams of Rheum tanguticum, 0.4-4.0 grams of Rehmannia glutinosa, and 0.2-2.0 grams of Lonicera japonica in about 4-40 grams of water or more, or in dry or lipidized form for oral, topical, sub-dermal or IV administration.

In another example, a known or discovered treatment regimen is supplemented with a low dose formula that includes 0.15-1.5 grams of Rheum tanguticum, 0.35-3.5 grams of Rehmannia glutinosa, and 0.15-1.5 grams of Lonicera japonica in about 3-30 grams of water or more, or in dry or lipidized form for oral, topical, sub-dermal or IV administration.

In another example, a known or discovered treatment regiment is supplemented with an herbal combination that includes 0.25-0.75 grams of Rheum tanguticum, 0.45-1.35 grams of Rehmannia glutinosa, and 0.25-0.75 grams of Lonicera japonica in about 25-75 grams of water, or in dry or lipidized form for oral, topical, sub-dermal or IV administration.

In another example, a known or discovered treatment regimen is supplemented with a low dose herbal formula that includes 0.33-1.0 grams of Rheum tanguticum, and 1.33-4 grams of Rehmannia glutinosa in about 10-30 grams of water, or in dry or lipidized form for oral, topical, sub-dermal or IV administration.

In another example, a known or discovered treatment regimen is supplemented with a high dose herbal formula that includes 0.30-1.0 grams of Rheum tanguticum and 0.6-2.0 grams of Rehmannia glutinosa in about 6-60 grams of water, or in dry or lipidized form for oral, topical, sub-dermal or IV administration.

In another example, a known or discovered treatment regimen is supplemented with a low dose herbal formula that includes 0.10-0.50 grams of Rheum tanguticum and 0.30-1.5 grams of Rehmannia glutinosa in about 4-20 grams of water, or in dry or lipidized form for oral, topical, sub-dermal or IV administration.

In another example, a known or discovered treatment regimen is supplemented with a low dose herbal formula that includes 0.20-0.50 grams of Rheum tanguticum and 0.40-1.0 grams of Rehmannia glutinosa in about 6-50 grams of water, or in dry or lipidized form for oral, topical, sub-dermal or IV administration.

In another example, a known or discovered treatment regimen is supplemented with a low dose herbal formula that includes 0.15-1.0 grams of Rheum tanguticum and 0.45-3.0 grams of Rehmannia glutinosa in about 6-40 grams of water, or in dry or lipidized form for oral, topical, sub-dermal or IV administration.

In another example, a known or discovered treatment regimen is supplemented with a low dose herbal formula that includes 0.25-0.75 grams of Rheum tanguticum in about 1-3 grams water, and 0.10-1.50 grams of Rehmannia glutinosa in about 1-5 grams of water, or in dry or lipidized form for oral, topical, sub-dermal or IV administration.

Examples of medium and high dose, and reduced and low dose herbal formulas for supplementing a known or discovered treatment regimen before, during or after administration of said known or discovered treatment regimen include one or more of the other herbs described herein, such as any of herbs contained in the seven herb and eighteen herb combinations described herein, that are administered in doses that are the same as or similar to or commensurate with the medium and high dose, and reduced and low dose example formulas for the one, two and three herb combinations described herein, including corresponding percentage changes when four or more herbs are included in the supplemental herbal formula that is administered before during or after a known or discovered treatment regimen. Many other combinations may be used depending on characteristics of the patient such as age and weight, the condition of the patient, and the patient's history.

Doses in between the low dose and high dose examples for the formulas that include one, two, or three or more herbs, or the seven herb formula, or the eighteen herb formula are also within the scope of further examples with per herb doses and/or total herb doses that are within the ranges provided in the examples above. Under certain conditions, doses above the high dose formula or below the low dose formula may be used as determined by a physician using his or her expertise and experience both generally in the field and with specific patients. In addition, other combinations of two, three, four, five, six or seven herbs, or more of these 18 herbs, and/or including one or more other herbs as understood by those skilled in the art, may be used in further examples of formulas wherein the dose ranges described in the above examples or otherwise as determined by a physician may be used. Further herbs not described herein may also be included in formulas including combinations of the described herbs and/or molecules, molecular extracts or molecular compounds described herein or understood by those skilled in the art.

Surprise effectiveness is also provided by very low dose herbal combinations of Rheum tanguticum, Rehmannia glutinosa and Lonicera japonica and by combinations of low doses of one or more of Rheum tanguticum, Rehmannia glutinosa and Lonicera japonica with certain herbal extracts, molecules and/or known medicines. Examples include combinations with methotrexate, carboplatin, vincristine, betamethasone, and dacarbazine.

In the examples above, certain doses of herb combinations have been described in certain amounts of water. Mixing the herbs in water is only one example of a way to take the herbal combinations. Instead, no water may be used and instead another liquid such as DMSO or an oil may be used. The herbs may be formed into pills or capsules, or a syringe or an IV may be used.

Any of the herbal combinations may be formed into a cream and rubbed onto the skin or hair, or a syringe may be used to inject a patient with an herbal combination.

In another example, the effect of CXCR4 antagonists on the survival of NB4, HL60, Jurkat leukemic cells and HT29 colon cancer and prostate tumor cells was examined. Digoxin significantly inhibited the growth of leukemic cells at concentrations between (0.05-to 1 microgram/ml). Emodin by itself inhibited the growth of leukemic cells only at concentrations of more than 5 microgrm/ml. Combinations of digoxin at concentrations of 0.1 microgram/ml and either 5 or 10 microgram/ml of emodin increased significantly the tumor killing ability of both compounds. Digoxin, Emodin, and their combination also add partial however significant effect on HT29 tumor cells.

Certain treatments may be prepared as a mixture of herbs that are known to have separately certain molecular components or medicinal advantages. For example, emodin may be extracted from traditional medicinal plants such as Rhei Rhizoma and Rheum Palmatum. In one embodiment, the source of Emodin used is Rheum tanguticum—Chinese name, or Rhubarb Root—English name, or Rheum Plamatum—Botanical name, or Radix Rhisoma Rhei—Pharmaceutical name. Emodin may be extracted from Rhubarb, Buckthorn and/or Japanese Knotweed (Fallopia Japonica). Aloe-emodin may be used which is a variety of emodin found in Socotrine, Barbados, and Zanzibar aloes.

A medicinal treatment is prepared in certain embodiments by mixing the herb, Rheum tanguticum, in water at a ratio of approximately 10:1. The herb may be ground to a fine powder. The water may be added to the fine powdered herb, and the pot covered. After boiling, the heat is lowered in certain embodiments to about 70 degrees centigrade. The aqueous mixture is cooked for another hour. The liquid is then strained into a container. In some cases, this may be done twice. In the second cooking, the ratio may be reduced to 7.5:1. The second cooking may take about 45 minutes including the boiling. Other herbs including Rehmannia glutinosa and/or Lonicera japonica may be mixed with the Rheum tanguticum or prepared alone or in combination with other herbs. Dry or lipidized capsules, or a cream, or a topical injection solution, or an IV solution, or other formulations understood to those skilled in the art may also be provided for administration of the treatment. The methotrexate or other known or described or discovered treatment that is administered before during and/or after the herbal medicinal treatment in accordance with certain embodiments may also be prepared in multiple ways as understood by those skilled in the art.

Single herbs or combinations of two or more herbs alone or with any one or more of the described molecules may be prepared in a process involving the following or a subset or variation thereof: grinding the herbs to a fine texture in the mixer for around 2-3 min until it looked fine powder; weighing the powder (e.g., 25 gm) and transferring to a beaker (e.g., 2000 ml); adding distilled water (RT) to powdered herbs in a ratio of 1:20 (gm of herbs: ml of water) and soaking the herbs for 15-20 min; boiling the mixture to 85-90° C.; cooling the temperature of the mixture down to 70-75° C. after removing it from the hot plate; covering the beaker properly with aluminum paper and cooking the mixture at 70° C. on hot plate-the total time of cooking of the mixture may be approximately 60 min which includes boiling, cooling it down and cooking; straining the mixture with the help of a manual strainer; after filtration, centrifuging the extract at 5000 rpm for 15 min and collecting the supernatant; filter sterilizing the supernatant by passing through 0.2 μm syringe filter; storing the clear filtrate at 4° C-subsequent dilutions may be prepared.

### Example Nanogel/Nano-Particulate Topical Formulation

An example nanogel formulation may include 1-20%, or or 1-15%, 1-10%, or 2.5-10% or 2.5-5% or approximately 2.5% or approximately 5% of an herbal mixture that includes Rheum tanguticum, Rehmannia glutinosa or Lonicera japonica or combinations thereof. An example nanogel formulation in accordance with certain embodiments may include fulvic acid, e.g., up to 1-10% or 2.5-10% or 2.5-5% or 1-5% or 5-10%.

An example nanogel or nano-particulate lotion, cream, ointment or shampoo formulation may include carbopol ultraze 21/polymer surfactant. Triethanolomine may be advantageously used to convert lotion to gel and/or to neutralize a level of ph to between 5-8, 5.5-7.5 or 6-7 for topical application.

Propylene glycol and/or polyethylene glycol may be included in a nanogel or other nano-particulate topical formulation, e.g., each 0%-15% each or 5%-10% each, or up to 15% total, or up to 20% total or up to a concentration level wherein spreadability may begin to become too low depending on other ingredients and topical administration considerations.

An example nanogel or other nano-particulate topical formulation may include DMDM Hydantoin.

A nano-liquid carrier may be included in a nano-particulate topical formulation in accordance with certain embodiments, e.g., stearic acid. The nano-particulate topical formulation may be lyophilized and homogenized with a dyno-mill, not exceeding a lipid limit, in an example embodiment.

Certain embodiments specifically do not include any parabins nor benzoid, as these have been deemed capable of effecting long term toxicity issues in certain patients or in a certain percentage of patients.

Hydrophyllic capacity is advantageously taken into account in certain embodiments such that permeability is greatly enhanced in a nanogel or other nano-particulate topical formulation including less than 450 nm particulates.

An example nano-particulate tablet formulation may include piperine, e.g., 1-10% or 2.5-10%, or 5-10% or 1-5% or 2.5-5%. A tablet formulation may be hardened above 40° C.-50° C. A nano-particulate tablet formulation may be reduced in size for ease of oral administration due to the enhanced permeability of nano-particulates compared with larger particulate sizes, e.g., above 450 nm. The permeability may be increased, e.g., from 10-20% to 20-80% or 30-70% or 40-60% or 50-60% and nano-particulates in accordance with certain embodiments advantageously may be packed stably into tablets of smaller sizes.

A nanogel or other nano-particulate topical formulation may include 1-20% or 1-15% or 1-10% or 2.5-10% or 2.5%-5% of the nano-particulate mixture. Nano-particulates may be 150-450 nm or 150-350 nm or 150-250 nm, or 100-300 nm or 100-400 nm or 100-450 nm or 50-450 nm, which serve to administer more of the medicine or medicinal supplement to a patient than the 10-20% permeability or lower of example formulations having 450 nm and above particulate sizes, because of reduced cumulative dose toxicities, better stability, and enhancement of treatment for the patient regarding tolerance and saturation issues.

A nano-emulsion, which tends to be oily, may include 100 nm or less particulate sizes.

A method of treating a skin or scalp condition in accordance with certain embodiments may include applying to the skin or scalp a medicinal composition formulated as a shampoo, conditioner, cream, ointment or other topical scalp or hair treatment that comprises a low concentration formula 5-10 grams or more of an active herbal combination of Rehmannia glutinosa, Rheum tanguticum and Lonicera japonica in an example 16 fluid ounce formulation. A high concentration formula may include 50-150 grams of the active herbal combination in an example 16 fluid ounce formulation. A medium concentration formula may include 10-50 grams of the active herbal combination in an example 16 fluid ounce formulation.

In certain embodiments, the medicinal composition may include 1%-20% of the active herbal combination. In certain embodiments, the medicinal composition may include 2.5%-10% of the active herbal combination. In certain embodiments, the medicinal composition may include 2.5%-5.0% of the active herbal combination. In certain embodiments, the medicinal composition may include 3.5 wt. % or more of Rheum tanguticum. In certain embodiments, the medicinal composition may include 15.4 wt. % or more of a combination of Rheum tanguticum and Rehmannia glutinosa.

In certain embodiments, the herbal combination may include 2-20 grams of Lonicera japonica, 2-20 grams of Rheum tanguticum and 6-60 grams of Rehmannia glutinosa. In certain embodiments, the medicinal composition may include 0.2 wt. %-4 wt. % of Lonicera japonica, 0.2 wt. %-4 wt. % of Rheum tanguticum, and 0.6 wt. %-12 wt. % of Rehmannia glutinosa.

In certain embodiments, the herbal combination may include 13.3-60 grams of Rehmannia glutinosa, 3.3-15 grams of Rheum tanguticum, and 3.3-15 grams of Lonicera japonica.

In certain embodiments, the combination of Rehmannia glutinosa, Rheum tanguticum and Lonicera japonica comprises 20 wt. %-80 wt. % of the active herbal combination. For example, the active herbal combination may include 12 wt. %-48 wt. % of Rehmannia glutinosa, 4 wt. %-16 wt. % of Rheum tanguticum and 4 wt. %-16 wt. % of Lonicera japonica.

In one example, a shampoo formulation may include between 2.5 wt. %-5.0 wt. % of a combination of Rehmannia glutinosa, Rheum tanguticum and Lonicera japonica.

In another example, a shampoo formulation may include between 1.0 wt. %-10.0 wt. % of a combination of Rehmannia glutinosa, Rheum tanguticum and Lonicera japonica.

In another example, a shampoo formulation may include between 1.0 wt. %-15.0 wt. % of a combination of Rehmannia glutinosa, Rheum tanguticum and Lonicera japonica.

A treatment regimen may include combinations of applications of shampoo, cream and/or lotion to affected scalp or other skin areas.

### Assessment of Wound Healing Potential of Test Items Using the Scratch Assay with Human Fibroblasts

The objective of this study was to assess the effect of test items on migration of human dermal fibroblast cells (HFF-1) on mechanically created scratch.

The present study assessed the wound healing efficiency of four herbal compositions, namely, Herb1, Herb2, Herb3 and 3HX (which was selected as 1:1:3 in this study and may be a combination of Herb1, Herb2 and Herb3 in a ratio in various example embodiments of approximately a:b:c, wherein 1<b/a<2, and 1<c/b<4) by evaluating their potential to aid in migration of human dermal fibroblast cells into the wounded monalayer using in vitro scratch assay method.

Confluent monolayers of HFF-1 cells were scratched horizontally and vertically to obtain linear wounds using sterile 200 μl pipette tip and incubated with the test items at the non-cytotoxic concentrations ranging from 1:500 to 1:50 (test item: cell growth medium). Thereafter, the ability of the cells to migrate and repopulate the scratched area was evaluated microscopically and photomicrographs were captured after 16 hours. The results were quantified using the image analyzer software, ImageJ.

The results obtained in the present study indicate that amongst all the four test items, only the three herb combination, or 3HX, demonstrated dose dependent wound healing potential at all the tested concentrations. However, none of the three herbs alone exhibited any significant potential as a fibroblast migration enhancer for improving wound healing efficiency across the tested concentrations.

### Test Item Information

### Test Item 1

Name of Test Item (TI): Herb 1
Scientific Name: Rheum tanguticum
Batch/Lot No.: D03-002
Plant part: Root and rhizome
Formulation type: Lyophilised powder
Yield: 4.0 mg/ml

### Test Item 2

Name of Test Item (TI): Herb2
Scientific Name: Lonicera japonica
Batch/Lot No.: J20140405
Plant part: Flower
Formulation type: Lyophilised powder
Yield: 5.6 mg/ml

### Test Item 3

Name of Test Item (TI): Herb3
Scientific Name: Rehmannia glutinosa
Batch/Lot No.: S20140513
Plant part: Root
Formulation type: Lyophilised powder
Yield: 26.6 mg/ml

### Test Item 4

Name of Test Item (TI): 3HX
Composition of TI: 3HX is a combination of roots of Herb 1, flowers of Herb2 and roots of Herb3 in 1:1:3 ratios
Batch/Lot No.: Herb 1-D03-002
   Herb2-J20140405
   Herb3-S20140513
Yield: 30 mg/ml
For all TIs
Test item analysis: Analysis for the identity and purity of the test item was not conducted as part of this study and is the responsibility of the sponsor

### Positive Control

Name of positive control (PC): Epidermal growth factor (EGF)
Catalog No.: PHG0311
Manufactured By: Gibco
Storage Condition: −20° C.

### Test System

Name: HFF-1 (Human dermal fibroblasts)
Source ATCC, USA
Growth Medium DMEM supplemented with 15%
FBS Growth Condition 37° C., 5% CO₂, 95% Humidity

A list of chemicals, reagents and media used in the study is provided in Table 1A. A list of test items and the amounts used in the study in milligrams is provided in Table 1B.

TABLE 1A
List of chemicals, reagents and the details of media

| Name of | | | |
|---|---|---|---|
| Chemical/Reagent | Manufacturer | Catalog No. | Batch/Lot No. |
| DMEM | Gibco | 12800-058 | 1481929 |
| DMSO | Merck | SG6S560543 | 80291205001730 |
| EDTA | Sigma | E5134 | SLBB5956V |
| FBS | GIBCO | 16000-044 | 1365340 |
| MTT | Sigma | M2128 | MKBH7489V |
| Penicillin/ | Himedia | A001A | 0000186078 |
| Streptomycin | | | |
| Sodium bicarbonate | Sigma | 55761 | 011M0147V |
| Trypsin | Sigma | T4799 | 016K7681 |

HFF-1 cells were maintained under conditions as described above and were subcultured by trypsinisation followed by splitting the cell suspension into fresh flasks and supplementing with fresh cell growth medium.

All four test items, namely Herb1, Herb2, Herb3, and 3HX, were dissolved in 1 ml of sterile MilliQ water as per their individual yield mentioned in table below to obtain weight/volume stocks which corresponded to 100% stock solution.

TABLE 1B

| S. No. | Name of Herb | Amount (in mg) |
|---|---|---|
| 1. | Herb1 | 4 |
| 2. | Herb2 | 5.6 |
| 3. | Herb3 | 26.6 |
| 4. | 3HX | 30 |

The stock solutions were then filtered through 0.22 μm syringe filter and diluted in DMEM (ie; cell growth medium) to obtain volume/volume concentrations corresponding to test item to cell growth medium ratios of 1:500, 1:200, 1:100, 1:75 and 1:50.

The concentrations obtained in the above step were used for subsequent treatment to the cells.

For formulation of a positive control, 100 μg/ml (PBS stock) of EGF was diluted in DMEM to achieve working concentrations ranging from 10 ng/ml—10,000 ng/ml (i.e., 10 fold or higher concentrations than the desired final concentrations in cells).

For determination of non-cytotoxic concentrations of test items and positive control (see Biological Evaluation of Medical Devices - Part 5: Tests for In Vitro Cytotoxicity (ISO 10993-5:2009), I.S.EN ISO, 10993-5:2009, which is incorporated by reference.), HFF-1 cells were counted using a haemocytometer and plated in a 96 well plate at the density corresponding to 5×103 cells/well/180 μl of DMEM supplemented with 15% FBS.

The above cells were then incubated overnight under growth conditions as described above so as to allow cell recovery and exponential growth.

After overnight incubation, the cells were subjected to serum starvation in DMEM so as to synchronize cell growth.

Following overnight incubation, the cells were treated with test items and positive control at the concentrations described above.

The above cells were then incubated for 16 hours in CO₂ incubator at 37° C., 5% CO₂ and 95% humidity.

Following incubation, the plates were taken out and 20 μl of 5 mg/ml of MTT [3-(4,5-dimethythiazol-2-yl)-2,5-diphenyl tetrazolium bromide] solution was added to all the wells followed by additional incubation for 3 h at 37° C. The supernatant was aspirated and 150 μl of DMSO was added to each well to dissolve formazan crystals. The absorbance of each well was read at 540 nm using Biotek Synergy HT micro plate reader.

The percentage cytotoxicity corresponding to each treatment was calculated using the following formula:

% Cytotoxicity=((R−X)/R)*100

Where X=Absorbance of treated cells at 540 nm
R=Absorbance of untreated cells at 540 nm

The concentrations exhibiting % cytotoxicity of <30% were considered as non-cytotoxic.

For assessment of the effect of test items on wound healing activity by scratch assay, HFF-1 cells were counted using hemocytometer and plated in 12 well plate at the density corresponding 0.08×10⁶ cells/well in DMEM supplemented with 15% FBS.

The above cells were then incubated overnight under growth conditions (e.g., 37° C., 5% CO₂, 95% Humidity), so as to allow the cell recovery and exponential growth.

Following overnight incubation, the cells were serum starved in DMEM so as to synchronize cell growth.

A small linear scratch (representative wound) was then created in the confluent monolayer by gently scraping horizontally and vertically with a sterile 200 μl micropipette tip.

The cells were then rinsed with serum free DMEM and grouped for treatment as follows: For baseline control, Cells plus DMEM were used. For positive/validation control, the baseline control plus EGF at non-cytotoxic concentrations (Stock solution serially diluted in serum free DMEM) were used. EGF was tested at the non-cytotoxic concentrations corresponding to 30 ng/ml. For tests 1-4, the baseline control plus the test items 1-4 of Table 1B. Test items 1-4 were each tested at five non-cytotoxic concentrations.

The above cells were then monitored for closure of wound area. Photomicrographs were captured after 16 hours and analyzed for quantitative assessment of area of wound closure using Image? software. The percentage migration corresponding to each treatment was calculated using the following formula:

% Migration=((R−X)/R)*100

Where X=% Scratched area of treated cells after 16 h, and
R=% Scratched area of untreated cells after 16 hours

### Results

The percentage cellular cytotoxicity obtained in HFF-1 cells treated with different concentrations of test items or positive control for 24 hours is represented in Table 2.

TABLE 2
Percentage cytotoxicity of test items in HFF-1 cells after 24 h of treatment

| | Test | % Cytotoxicity wrt |
|---|---|---|
| Treatment | Concentration | untreated cells |
| EGF (ng/ml) | 10 | −17.56 |
| | 30 | −12.19 |
| | 100 | −3.60 |
| Herb1 | 1:500 | −7.67 |
| | 1:200 | 0.40 |
| | 1:100 | −13.67 |
| | 1:75 | 8.64 |
| | 1:50 | 18.48 |
| | 1:25 | 30.61** |
| Herb2 | 1:500 | −2.57 |
| | 1:200 | −11.67 |
| | 1:100 | 12.47 |
| | 1:75 | −6.06 |
| | 1:50 | −12.07 |
| | 1:25 | −10.64 |
| Herb3 | 1:500 | 19.39 |
| | 1:200 | 18.76 |
| | 1:100 | 20.71 |
| | 1:75 | 23.28 |
| | 1:50* | 27.66 |
| | 1:25 | 33.70** |
| 3HX | 1:500 | −0.92 |
| | 1:200 | 18.48 |
| | 1:100 | 19.22 |
| | 1:75 | 20.42 |
| | 1:50 | 23.11 |
| | 1:25 | 30.78** |

Each value represents mean of triplicate wells
*Value represents mean of duplicate wells.
**30% cytotoxicity

In view of the results shown in Table 2, amongst the four test items, Herb1, Herb3 and 3HX demonstrated >30% cytotoxicity at the highest concentration corresponding to 1:25. Therefore, the same were not tested for wound healing efficiency. All the four test items were tested for their effect on migration of HFF-1 cells towards the wounded site at the concentrations ranging from 1:500, 1:200, 1:100, 1:75 and 1:50.

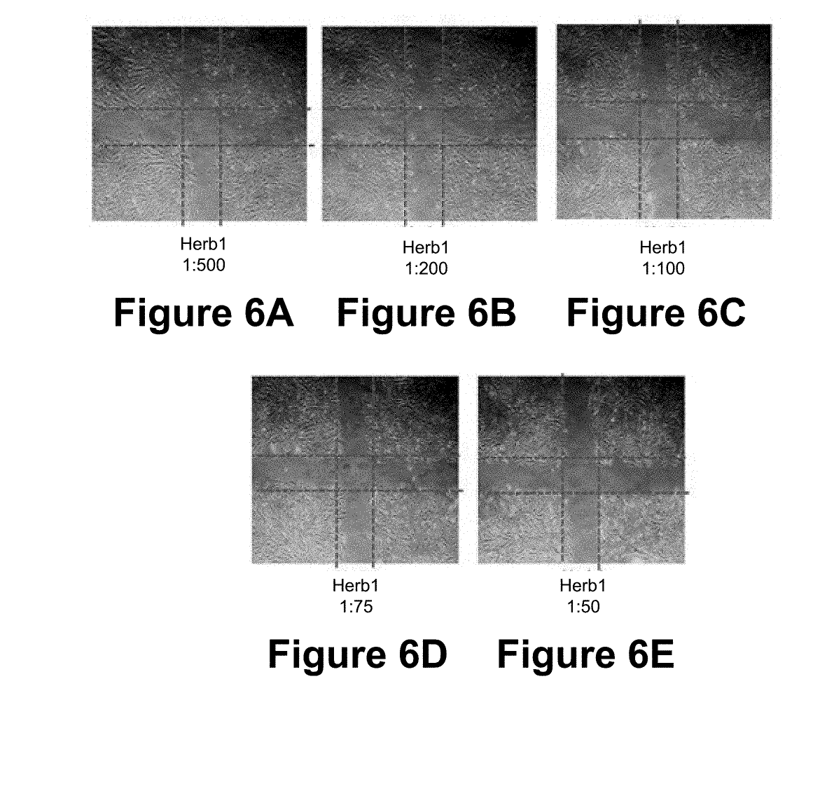

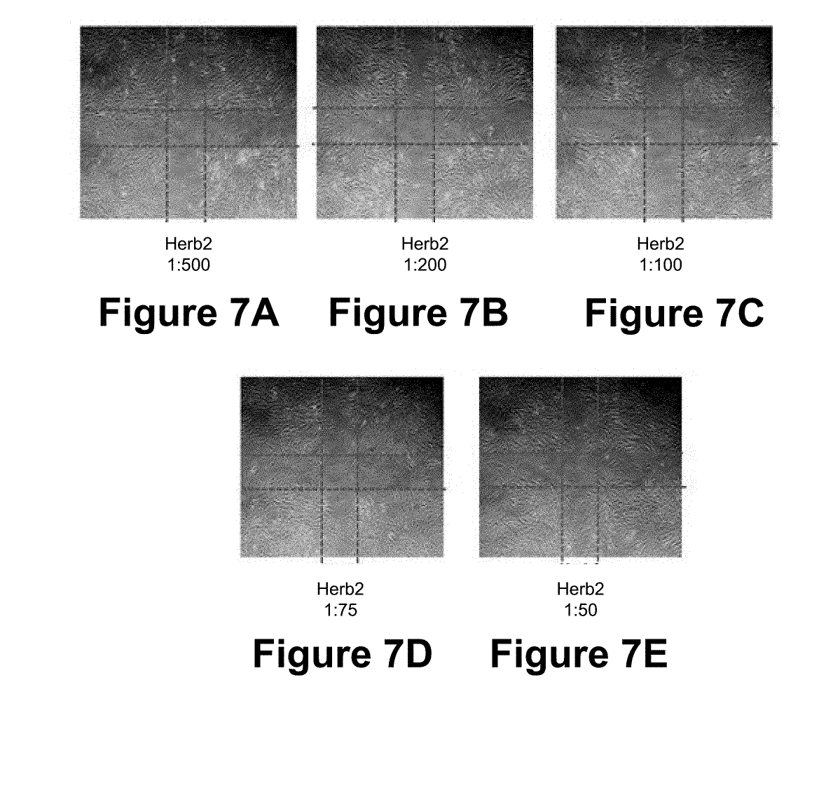

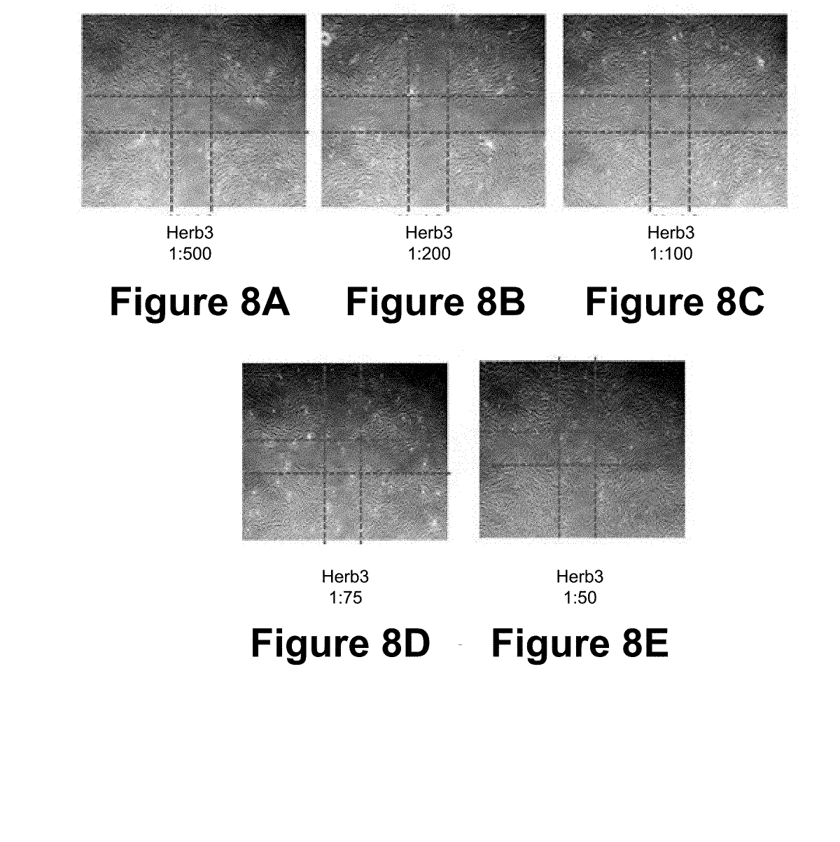

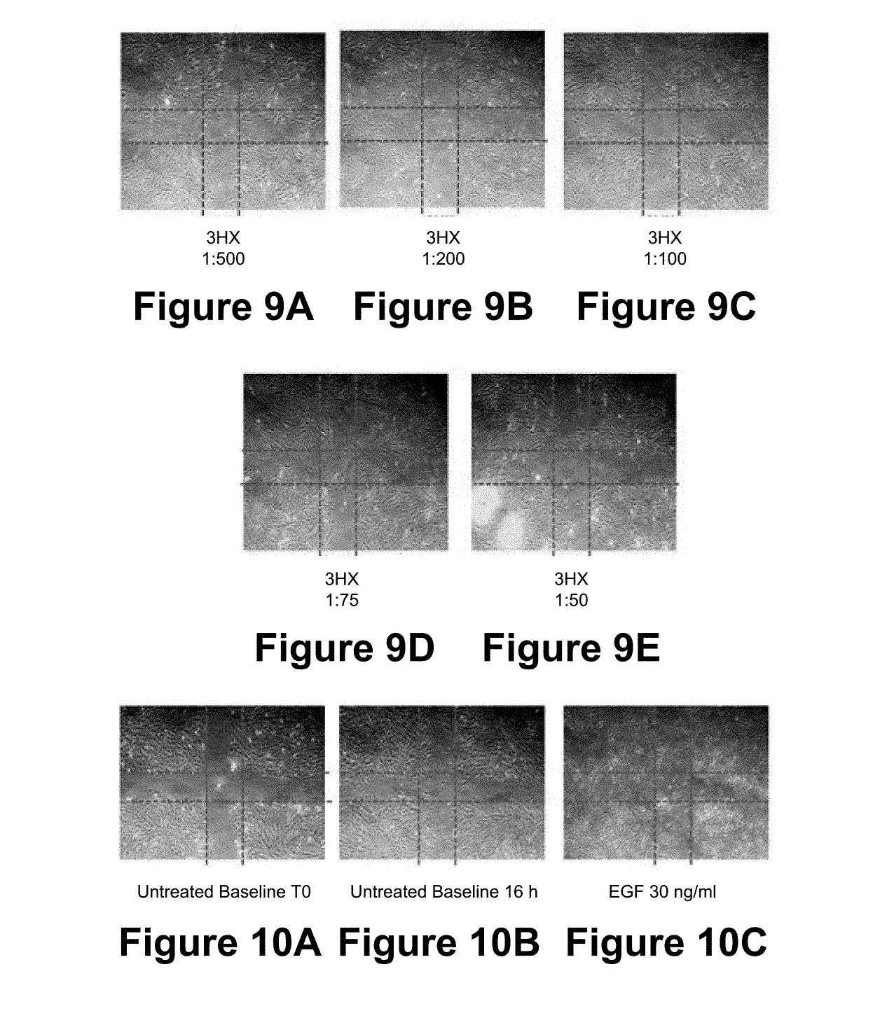

The effect of migration corresponding to each treatment was assessed based on the extent of wound closure of HFF-1 cells after 16 hours of treatment. The detailed results are provided in FIGS. 6A-6E for Herb 1, FIGS. 7A-7E for Herb 2, FIGS. 8A-8E for Herb 3 and FIGS. 9A-9E for the combination of Herb 1, Herb 2 and Herb 3 at ratio 1:1:3 in an example embodiment. These results are summarized in Table 3.

TABLE 3
Quantitative wound healing effect of test
items in HFF-1cells after 16 h of treatment

| | | Test | % Wound healing efficiency wrt |
|---|---|---|---|
| Treatment | | Concentration | untreated (t = 16 h) |
| | Untreated | | 0 |
| | EGF (ng/ml) | 30 | 53.96 |
| | Herb1 | 1:500 | −53.81 |
| | | 1:200 | −49.09 |
| | | 1:100 | −74.12 |
| | | 1:75 | −62.03 |
| | | 1:50 | −92.05 |
| | Herb2 | 1:500 | −43.43 |
| | | 1:200 | −30.40 |
| | | 1:100 | −53.83 |
| | | 1:75 | −20.50 |
| | | 1:50 | −30.16 |
| | Herb3 | 1:500 | −47.67 |
| | | 1:200 | −66.77 |
| | | 1:100 | −7.65 |
| | | 1:75 | −9.36 |
| | | 1:50 | −2.12 |
| | 3HX | 1:500 | 30.34 |
| | | 1:200 | 29.84 |
| | | 1:100 | 19.88 |
| | | 1:75 | 9.27 |
| | | 1:50 | 7.62 |

The present study was conducted to assess the wound healing potential of four herbal formulations, namely Herb1, Herb2, Herb3 and 3HX, which is a combination of Herb1, Herb2 and Herb3 in a ratio a:b:c where 1<b/a<2 and 1<c/b<4, and in certain example embodiments in ratios 1:1:2, 1:1:2.5, 1:1:3, and 1:1:4. Human dermal fibroblast cells, HFF-1 were used as test system in the present study as these cells play a key role in wound re-epithelialization. Briefly, confluent monolayer of cells were scratched to generate a mechanical wound followed by treatment with the test items at volume/volume concentrations corresponding to test item to cell growth medium ratios of 1:500, 1:200, 1:100, 1:75 and 1:50 for 16 hours. The cells were visualized under inverted microscope for closure of wound. Thereafter, the cells were photomicrographed and the images were analysed using ImageJ software so as to quantitate the wound healing effect of the herbs.

The wound healing effect of Herb1 is shown in FIGS. 6A-6E and Table 3. When compared with untreated cells, Herb 1 did not demonstrate any wound healing effect.

The wound healing effect of Herb2 is shown in FIGS. 7A-7E and Table 3. When compared with untreated cells, Herb2 did not demonstrate any wound healing effect.

The wound healing effect of Herb3 is shown in FIGS. 8A-8E and Table 3. When compared with untreated cells, Herb3 did not demonstrate any wound healing effect.

The wound healing effect of 3HX (i.e., the combination of Herb 1, Herb 2 and Herb 3 at a ratio 1:1:3 in this example) is shown in FIGS. 9A-9E and Table 3. When compared with untreated cells, the three herb combination (3HX) demonstrated dose dependent enhanced migration of human dermal fibroblasts towards scratched area across all the tested concentrations. The quantitative analysis revealed that the herb led to an average of 19.39% increase in migration of dermal fibroblasts towards the scratched area as compared to the untreated cells after 16 hours. The overall effect ranged from 7.62% (at 1:50) to 30.34% (at 1:500) increase in migration of 3HX treated and wounded dermal fibroblast cells. The findings from the present study indicate that 3HX led to an increase in migration of human dermal fibroblast cells at all the tested concentrations.

FIG. 10A illustrates a baseline untreated wound at the start of a HFF-1 cell migration test.

FIG. 10B illustrates the baseline untreated wound of FIG. 10A after 16 hours.

FIG. 10C illustrates effects of the EGF baseline control solution on migration of HFF-1 cells after 16 hours.

### Assessment of Wound Healing Potential of Test Items Using the Scratch Assay with Human Keratinocytes

The objective of this study was to assess the effect of test items on migration of human keratinocytes (HaCaT) on a mechanically created scratch. The present study assessed the wound healing efficiency of four herbs viz., Herb 1, Herb2, Herb3 and 3HX by evaluating their potential to aid in migration of human dermal fibroblast cells into the wounded monalayer using in vitro scratch assay method. Briefly, the confluent monolayers of HACAT cells were scratched horizontally and vertically to obtain a linear wounds using sterile 200 μl pipette tip and incubated with the test items at the non-cytotoxic concentrations ranging from 1: 500 to 1:50 (test item : cell growth medium). Thereafter, the ability of the cells to migrate and repopulate the scratched area was evaluated microscopically and photomicrographs were captured after 16 hours. The results were quantified using the image analyzer software, ImageJ. The results obtained in the present study indicate that amongst all the herbs, only Herb2 demonstrated wound healing potential at all the concentrations corresponding to 1:500, 1:200, 1:100 and 1:75. However, the other three herbs namely, Herb 1, Herb3 and 3HX did not exhibit any wound healing potential across the tested concentrations.

### Test Item Information

### Test Item 1

Name of Test Item (TI): Herb1
Scientific Name: Rheum tanguticum
Batch/Lot No.: D03-002
Plant part: Root and rhizome
Formulation type: Lyophilised powder
Yield: 4.0 mg/ml

### Test Item 2

Name of Test Item (TI): Herb2
Scientific Name Lonicera japonica
Batch/Lot No.: J20140405
Plant part: Flower
Formulation type: Lyophilised powder
Yield: 5.6 mg/ml

### Test Item 3

Name of Test Item (TI): Herb3
Scientific Name: Rehmannia glutinosa
Batch/Lot No.: S20140513
Plant part: Root
Formulation type: Lyophilised powder
Yield: 26.6 mg/ml

### Test Item 4

Name of Test Item (TI): 3HX
Composition of TI: 3HX is a combination of roots of Herb 1, flowers of Herb2 and roots of Herb3 in 1:1:3 ratios
Batch/Lot No.: Herb 1-D03-002
   Herb2-J20140405
   Herb3-S20140513
Yield: 30 mg/ml
For all TIs
Test item analysi:s Analysis for the identity and purity of the test item was not conducted as part of this study and is the responsibility of the sponsor

### Positive Control

Name of positive control (PC): Epidermal growth factor (EGF)
Catalog No.: PHG0311
Manufactured By: Gibco
Storage Condition: −20° C.

### Test System

Name: HaCaT (Human keratinocytes)
Source: NCCS, Pune
Growth Medium: DMEM supplemented with 10% FBS
Growth Condition: 37° C., 5% CO₂, 95% Humidity

On the basis of above information, in order to meet the objective of the present study, HaCaT (Human keratinocytes), cell line were used as test system.

A list of chemicals and reagents and details of media used in the study is provided at Table 1A. A list of test items and the amounts used in the study in milligrams is provided in Table 1B.

HaCaT cells were subcultured by trypsinisation followed by splitting the cell suspension into fresh flasks and supplementing with fresh cell growth medium.

All the four test items namely Herb1, Herb2, Herb3, and 3HX (i.e., a combination of Herb 1, Herb 2 and Herb 3 in a ratio 1:1:3 in an example embodiment) were dissolved in 1 ml of sterile MilliQ water as per their individual yield mentioned in table below to obtain weight/volume stocks which corresponded to 100% stock solution.

The stock solutions were then filtered through 0.22 μm syringe filter and diluted in DMEM (ie; cell growth medium) to obtain volume/volume concentrations corresponding to test item to cell growth medium ratios of 1:500, 1:200, 1:100, 1:75 and 1:50.

The concentrations obtained in the above step were used for subsequent treatment to the cells.

For formulation of the positive control, 100 μg/ml (PBS stock) of EGF was diluted in DMEM to achieve working concentrations ranging from 10 ng/ml-10,000 ng/ml (10 fold high concentrations than the desired final concentrations in cells).

Determination of non-cytotoxic concentrations of test items and positive control (see Biological Evaluation of Medical Devices—Part 5: Tests for In Vitro Cytotoxicity (ISO 10993-5:2009), I.S.EN ISO, 10993-5:2009, which is incorporated by reference.)

HaCaT cells were counted using a haemocytometer and plated in a 96 well plate at the density corresponding to 5×103 cells/well/180W of DMEM supplemented with 10% FBS.

The above cells were then incubated overnight under growth conditions as described above so as to allow cell recovery and exponential growth.

After overnight incubation, the cells were subjected to serum starvation in DMEM so as to synchronize cell growth.

Following overnight incubation, the cells were treated with test item and positive control at the concentrations as described above.

The above cells were then incubated for 24 h in CO₂ incubator at 37° C., 5% CO₂ and 95% humidity.

Following incubation, the plates were taken out and 20 μl of 5 mg/ml of MTT [3-(4,5-dimethythiazol-2-yl)-2,5-diphenyl tetrazolium bromide] solution was added to all the wells followed by additional incubation for 3 h at 37° C. The supernatant was aspirated and 150 μl of DMSO was added to each well to dissolve formazan crystals. The absorbance of each well was read at 540 nm using Biotek Synergy HT micro plate reader.

The percentage cytotoxicity corresponding to each treatment was calculated using the following formula:

% Cytotoxicity=((R−X)/R)*100

Where X=Absorbance of treated cells at 540 nm and
R=Absorbance of untreated cells at 540 nm

The concentrations exhibiting % cytotoxicity of <30% were considered as non-cytotoxic.

For assessment of the effect of test items on wound healing activity by scratch assay, HaCaT cells were counted using hemocytometer and plated in 12 well plate at the density corresponding 0.2×10⁶ cells/well in DMEM supplemented with 10% FBS.

The above cells were then incubated overnight under growth conditions as described above so as to allow the cell recovery and exponential growth.

Following overnight incubation, the cells were serum starved in DMEM so as to synchronize cell growth.

A small linear scratch (representative wound) was then created in the confluent monolayer by gently scraping horizontally and vertically with a sterile 200 μl micropipette tip.

The cells were then rinsed with serum free DMEM and grouped for treatment as follows: For baseline control, Cells plus DMEM were used. For positive/validation control, the baseline control plus EGF at non-cytotoxic concentrations (Stock solution serially diluted in serum free DMEM) were used. EGF was tested at the non-cytotoxic concentrations corresponding to 30 ng/ml. For tests 1-4, the baseline control plus the test items 1-4 of Table 1B. Test items 1-4 were each tested at five non-cytotoxic concentrations.

The above cells were then monitored for closure of wound area.

Photomicrographs were captured after 24 hours and analyzed for quantitative assessment of area of wound closure using ImageJ software.

The percentage migration corresponding to each treatment was calculated using the following formula:

% Migration=((R−X)/R)*100

Where X=% Scratched area of treated cells after 24 hours and
R=% Scratched area of untreated cells after 24 hours.

### Results

The percentage cellular cytotoxicity obtained in HaCaT cells treated with different concentrations of test items or positive control for 24 h is represented in Table 4. Amongst the four test items only Herb1 demonstrated >30% cytotoxicity at the highest concentration corresponding to 1:25. Therefore, the same was not tested for wound healing efficiency. All four test items were tested for their effect on migration of HaCaT cells towards the wounded site at the concentrations ranging from 1:500, 1:200, 1:100, 1:75 and 1:50.

TABLE 4
Percentage cytotoxicity of test items in HaCaT cells after 24 h of treatment

| | Test | % Cytotoxicity wrt |
|---|---|---|
| Treatment | Concentration | untreated cells |
| EGF (ng/ml) | 10 | −15.95 |
| | 30 | −11.79 |
| | 100 | −1.53 |
| Herb1 | 1:500 | 0.32 |
| | 1:200 | 8.12 |
| | 1:100 | −3.99 |
| | 1:75 | −4.77 |
| | 1:50 | −1.25 |
| | 1:25 | 50.21** |
| Herb2 | 1:500 | 1.53 |
| | 1:200 | 5.56 |
| | 1:100 | 1.67 |
| | 1:75 | 3.06 |
| | 1:50 | 9.72 |
| | 1:25 | 8.12 |
| Herb3 | 1:500 | 7.62 |
| | 1:200 | 6.73 |
| | 1:100 | 3.74 |
| | 1:75 | 12.68 |
| | 1:50 | 22.22 |
| | 1:25 | −0.32 |
| 3HX | 1:500 | 5.27 |
| | 1:200 | −12.93 |
| | 1:100 | −19.98 |
| | 1:75 | 9.47 |
| | 1:50 | 8.51 |
| | 1:25 | 11.08 |

Each value represents mean of triplicate wells
**30% cytotoxicity

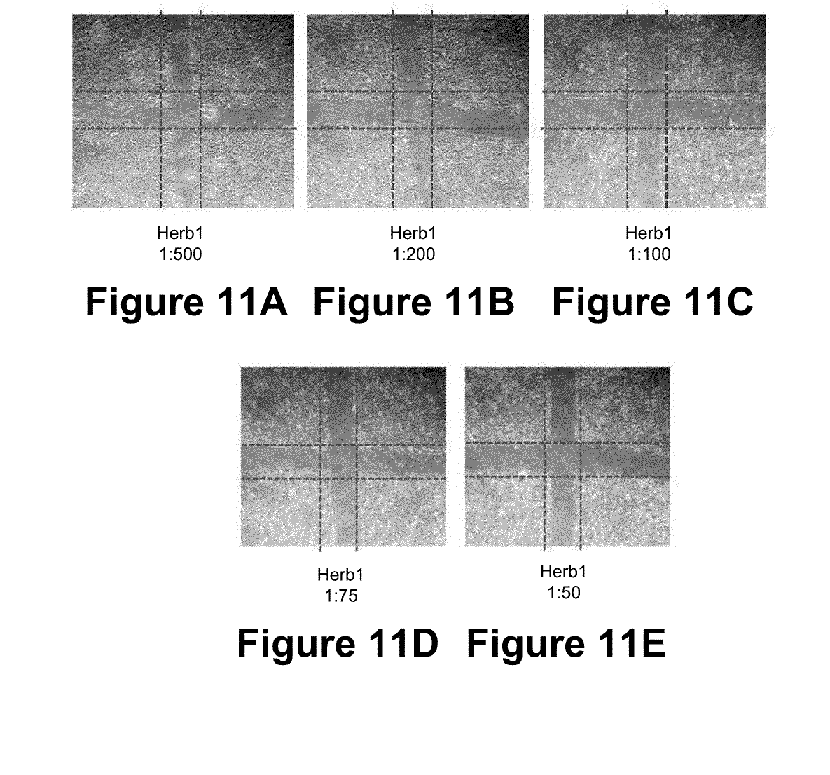

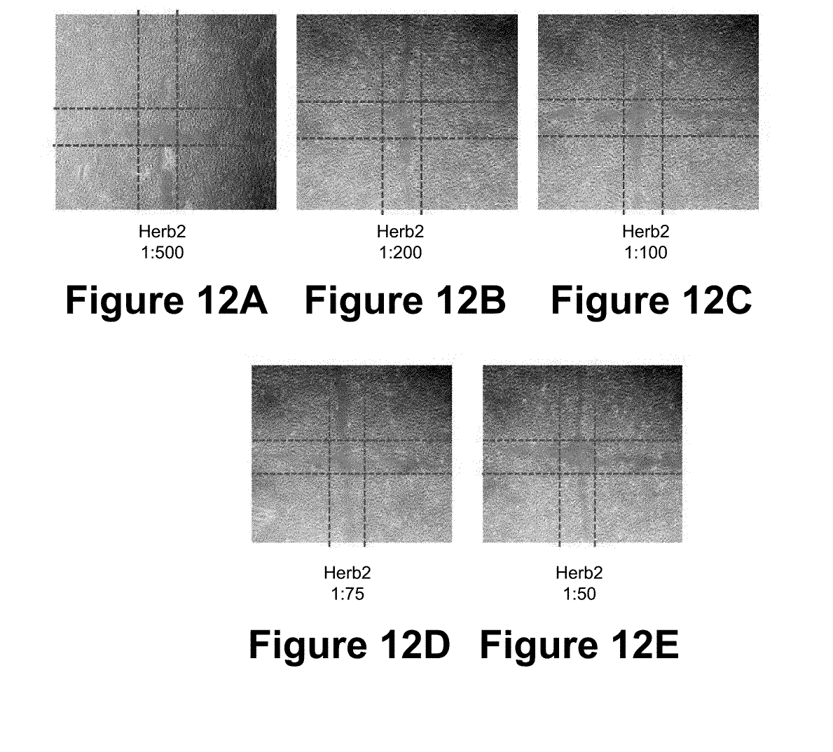

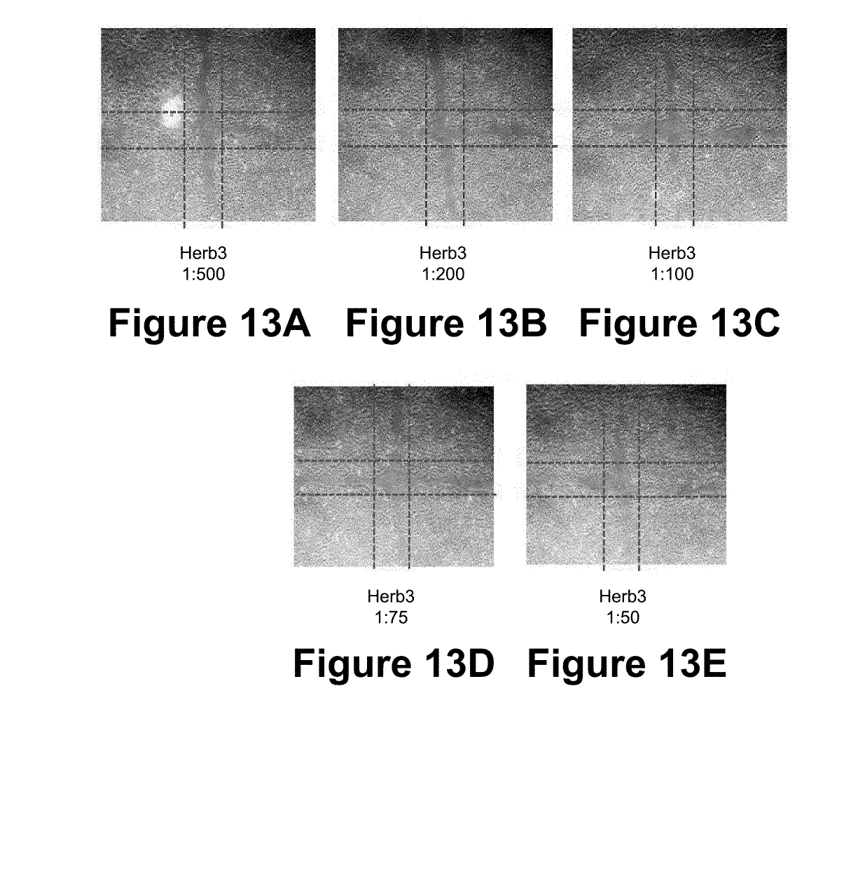

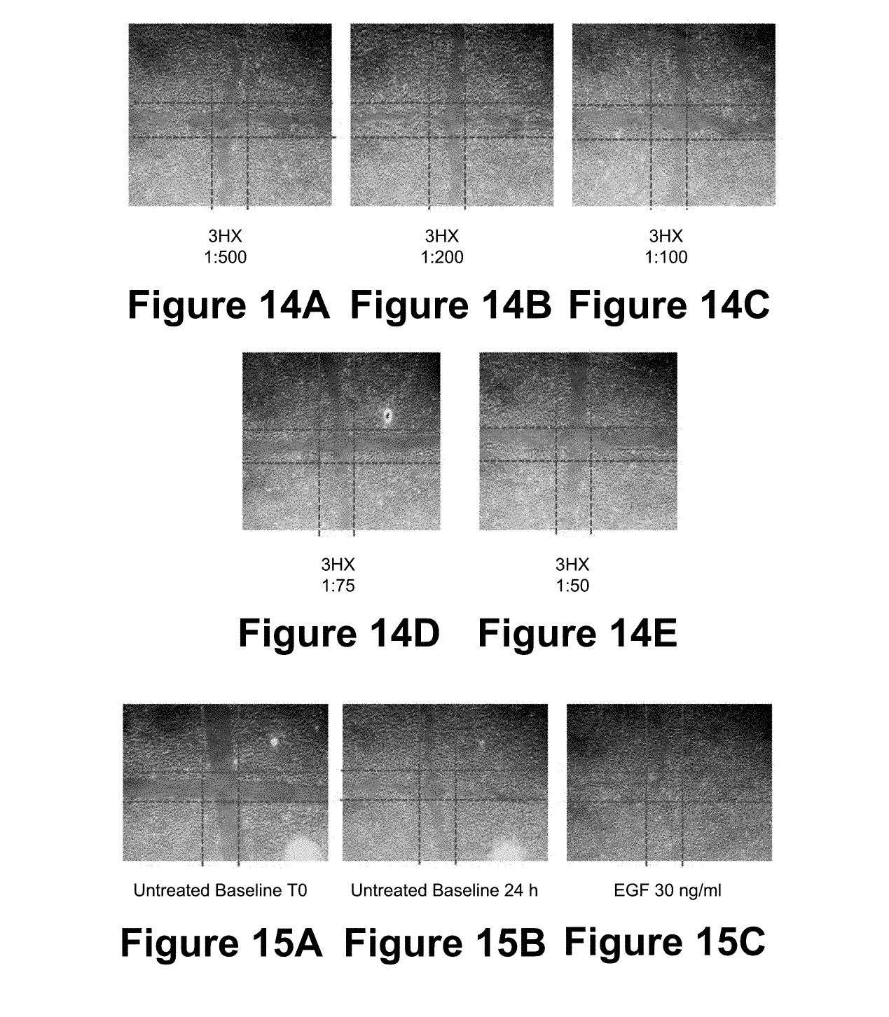

The effects of test items on migration of HaCaT cells (Scratch assay) corresponding to each treatment was assessed based on the extent of wound closure of HaCaT cells after 24 hours of treatment. The detailed results are provided in FIGS. 11A-11E for Herb 1, FIGS. 12A-12E for Herb 2, FIGS. 13A-13E for Herb 3 and FIGS. 14A-14E for the combination of Herb 1, Herb 2 and Herb 3 at ratio 1:1:3 in an example embodiment. These results are summarized in Table 5.

TABLE 5
Quantitative wound healing effect of test
items in HaCaT cells after 24 h of treatment

| | Test | % Wound healing efficiency |
|---|---|---|
| Treatment | Concentration | wrt untreated (t = 24 h) |
| Untreated | | 0 |
| EGF (ng/ml) | 30 | 61.9** |
| Herb1 | 1:500 | −80.3 |
| | 1:200 | −145.8 |
| | 1:100 | −129.6 |
| | 1:75 | −131.0 |
| | 1:50 | −168.4 |
| Herb2 | 1:500 | 46.8** |
| | 1:200 | 26.3** |
| | 1:100 | 15.8** |
| | 1:75 | 5.9** |
| | 1:50 | −19.9 |
| Herb3 | 1:500 | −2.3 |
| | 1:200 | −1.0 |
| | 1:100 | −0.4 |
| | 1:75 | −28.0 |
| | 1:50 | −2.8 |
| 3HX | 1:500 | −34.1 |
| | 1:200 | −31.3 |
| | 1:100 | −43.6 |
| | 1:75 | −50.7 |
| | 1:50 | −86.2 |

The present study was conducted to assess the wound healing potential of four herbs viz., Herb1, Herb2, Herb3 and 3HX. Human keratinocyte cells, HaCaT were used as test system in the present study as these cells play a key role in wound re-epithelialization. Briefly, confluent monolayer of cells were scratched to generate a mechanical wound followed by treatment with the test items at volume/volume concentrations corresponding to test item to cell growth medium ratios of 1:500, 1:200, 1:100, 1:75 and 1:50 for 24 h. The cells were visualized under inverted microscope for closure of wound. Thereafter, the cells were photomicrographed and the images were analysed using ImageJ software so as to quantitate the wound healing effect of the herbs.

The Wound healing effect of Herb1 is shown in FIGS. 11A-11E and Table 5. When compared with untreated cells, Herb 1 did not demonstrate any wound healing effect.

The Wound healing effect of Herb2 is shown in FIGS. 12A-12E and Table 5. When compared with untreated cells, Herb2 demonstrated wound healing effect towards scratched area at the concentrations ranging from 1:500 to 1:75. The overall effect ranged from 5.9% (at 1:75) to 32.5% (at 1:500) increase in migration of Herb2 treated and wounded epidermal keratinocyte cells. However, no effect in wound healing was observed at the highest concentration of 1:50.

The Wound healing effect of Herb3 is shown in FIGS. 13A-13E and table 5. When compared with untreated cells, Herb 3 did not demonstrate any wound healing effect.

The Wound healing effect of 3HX (i.e., the three herb combination of Herb 1, Herb 2 and Herb 3 at 1:1:3 in certain example embodiments) is shown in FIGS. 14A-14E and Table 5

. When compared with untreated cells, 3HX did not demonstrate any wound healing effect.

The findings of the present study indicate that out of the four herbs tested, only Herb2 led to an increase in migration of human epidermal keratinocyte cells.

FIG. 15A illustrates a baseline untreated wound at the start of a HaCaT cell migration test.

FIG. 15B illustrates the baseline untreated wound of FIG. 15A after 24 hours.

FIG. 15C illustrates effects of the EGF baseline control solution on migration of HaCaT cells after 24 hours.

The following is incorporated by reference: Walter M. et al.; Mesenchymal stem cell-conditioned medium accelerates skin wound healing: An in vitro study of fibroblast and keratinocyte scratch assays. Experimental cell research. 2010; 316:1271-1281.

While the invention has been described in terms of several embodiments, those skilled in the art will recognize that the invention is not limited to the embodiments described, but can be practiced with modification and alteration within the spirit and scope of the appended claims. The description is thus to be regarded as illustrative instead of limiting of the invention as set forth in the appended claims including structural and functional equivalents thereof

### Abbreviations Used

| ABBREVIATION | DESCRIPTION |
|---|---|
| ° C. | Degree centigrade |
| DMEM | Dulbecco's Modified Eagle's Medium |
| DMSO | Dimethyl sulphoxide |
| ECM | Extracellular matrix |
| EGF | Epidermal growth factor |
| FBS | Fetal bovine serum |
| g | Acceleration due to gravity |
| h | Hour |
| μg | Microgram |
| μl | Microlitre |
| min | Minutes |
| ml | Millilitre |
| MTT | 3-(4,5-Dimethylthiazol-2-yl)-2, Diphenyltetrazolium |
| | bromide |
| nm | Nanometer |
| NCCS | National Centre for Cell Science |
| OD | Optical Density |
| PBS | Phosphate Buffer Saline |
| RH | Relative Humidity |
| RT | Room Temperature |
| TI | Test Item |

What is claimed is:

1. A method of enhancing fibroblast cell migration and skin wound healing efficiency, comprising administering to a patient with a skin wound a dosage composition comprising a fibroblast cell migration enhancing active component that includes at least 60 wt. % of an herbal combination consisting of between three and seven herbs at least including rheum tanguticum, lonicera japonica, and rehmannia glutinosa.

2. The method of claim 1, comprising diluting said active component between 1:50 and 1:500 in an aqueous or other inactive solution configured for topical application at a location of said skin wound.

3. The method of claim 1, wherein said dosage composition comprises at least 30 mg of said herbal combination.

4. The method of claim 3, wherein said dosage composition comprises at least 20.4 mg of said rehmannia glutinosa, at least 4 mg of said rheum tanguticum, and at least 5.6 mg of said lonicera japonica.

5. The method of claim 1, wherein said active component comprises at least 30 mg/ml of said herbal combination.

6. The method of claim 5, wherein said active component comprises at least 20.4 mg/ml of said rehmannia glutinosa, at least 4 mg/ml of said rheum tanguticum, and at least 5.6 mg/ml of said lonicera japonica.

7. The method of claim 5, wherein said dosage composition comprises between 60 ng/ml and 600 ng/ml of said herbal combination diluted in an aqueous or other inactive solution between 1:50 and 1:500.

8. The mGethod of claim 1, comprising repeating said administering of said dosage composition multiple times in a treatment regimen that includes administering over a duration of said treatment regimen at least 13.3 grams of the rehmannia glutinosa, at least 3.3 grams of the rheum tanguticum, and at least 3.3 grams of the lonicera japonica.

9. The method of claim 8, comprising topically administering the dosage composition at a location of said skin wound site multiple times over said duration of said treatment regimen.

10. The method of claim 1, comprising reducing an average particulate size of the active component to less than 450 nm, and combining said active component with one or more inactive ingredients to produce a nanogel formulation configured for direct application at a location of said skin wound.

11. The method of claim 1, comprising reducing an average particulate size of the active component to less than 350 nm, and combining said active component with one or more inactive ingredients to produce a nanogel formulation configured for direct application at a location of said skin wound.

12. The method of claim 1, comprising reducing an average particulate size of the active component to less than 250 nm, and combining said active component with one or more inactive ingredients to produce a nanogel formulation configured for direct application at a location of said skin wound.

13. The method of claim 1, comprising administering a methotrexate regimen before, during, or after administrating said dosage composition, or combinations thereof

14. The method of claim 1, comprising combining said active component with one or more inactive ingredients to produce a cream, lotion, ointment or other topical formulation configured for direct application at a location of said skin wound.

15. The method of claim 14, comprising diluting said active component within said topical formulation between 1:50 and 1:500.

16. The method of claim 1, comprising combining said active component with one or more inactive ingredients to produce a shampoo, conditioner, or other topical scalp or hair treatment.

17. A method of formulating a dosage composition for enhancing fibroblast cell migration and skin wound healing efficiency, comprising formulating a fibroblast cell migration enhancing active component that includes at least 60 wt. % of an herbal combination consisting of between three and seven herbs at least including rheum tanguticum, lonicera japonica, and rehmannia glutinosa.

18. The method of claim 17, comprising combining said active component with one or more inactive ingredients to produce a cream, lotion, ointment or other topical formulation configured for direct application at a location of said skin wound.

19. The method of claim 18, comprising diluting said active component within said topical formulation between 1:50 and 1:500.

20. The method of claim 17, comprising combining said active component with one or more inactive ingredients to produce a shampoo, conditioner, or other topical scalp or hair treatment.

21. The method of claim 17, comprising reducing an average particulate size of the active component to less than 450 nm, and combining said active component with one or more inactive ingredients to produce a nanogel formulation configured for direct application at a location of said skin wound.

22. The method of claim 17, comprising reducing an average particulate size of the active component to less than 350 nm, and combining said active component with one or more inactive ingredients to produce a nanogel formulation configured for direct application at a location of said skin wound.

23. The method of claim 17, comprising reducing an average particulate size of the active component to less than 250 nm, and combining said active component with one or more inactive ingredients to produce a nanogel formulation configured for direct application at a location of said skin wound.

24. The method of claim 17, comprising formulating a combination of methotrexate with said dosage composition for administration before, during, or after administration of said dosage composition, or combinations thereof.

25. A dosage composition for enhancing fibroblast cell migration and skin wound healing efficiency, comprising a fibroblast cell migration enhancing active component that includes at least 60 wt. % of an herbal combination consisting of between three and seven herbs at least including rheum tanguticum, lonicera japonica, and rehmannia glutinosa.

26. The dosage composition of claim 25, wherein said active component is diluted in an aqueous or other inactive solution between 1:50 and 1:500.

27. The dosage composition of claim 25, comprising at least 30 mg of said herbal combination.

28. The dosage composition of claim 27, comprising at least 20.4 mg of said rehmannia glutinosa, at least 4 mg of said rheum tanguticum, and at least 5.6 mg of said lonicera japonica.

29. The dosage composition of claim 25, wherein said active component comprises at least 30 mg/ml of said herbal combination.

30. The dosage composition of claim 29, wherein said active component comprises at least 20.4 mg/ml of said rehmannia glutinosa, at least 4 mg/ml of said rheum tanguticum, and at least 5.6 mg/ml of said lonicera japonica

31. The dosage composition of claim 29, comprising between 60 ng/ml and 600 ng/ml of said herbal combination diluted in an aqueous or other inactive solution between 1:50 and 1:500.

32. The dosage composition of claim 25, comprising a cream, lotion, ointment or other topical formulation configured for direct application at a location of said skin wound.

33. The dosage composition of claim 32, wherein said active component is diluted within said topical formulation between 1:50 and 1:500.

34. The dosage composition of claim 25, comprising a shampoo, conditioner, or other topical scalp or hair treatment.

35. The dosage composition of claim 25, wherein said active component comprises particulates of reduced average size to less than 450 nm.

36. The dosage composition of claim 35, comprising one or more inactive ingredients combined with said particulates of said active component to produce a nanogel formulation configured for direct application at a location of said skin wound.

37. The dosage composition of claim 25, wherein said active component comprises particulates of reduced average size to less than 350 nm.

38. The dosage composition of claim 37, comprising one or more inactive ingredients combined with said particulates of said active component to produce a nanogel formulation configured for direct application at a location of said skin wound.

39. The dosage composition of claim 25, wherein said active component comprises particulates of reduced average size to less than 250 nm.

40. The dosage composition of claim 37, comprising one or more inactive ingredients combined with said particulates of said active component to produce a nanogel formulation configured for direct application at a location of said skin wound.

41. The dosage composition of claim 25, comprising a combination of methotrexate with said dosage composition for administration before, during, or after administration of said dosage composition, or combinations thereof.

42. A treatment regimen that includes multiple doses of a composition for enhancing fibroblast cell migration and skin wound healing efficiency, comprising a housing containing said multiple doses that each include a fibroblast cell migration enhancing active component comprising at least 60 wt. % of an herbal combination consisting of between three and seven herbs at least including rheum tanguticum, lonicera japonica, and rehmannia glutinosa.

43. The treatment regimen of claim 42, wherein said multiple doses each contain at least 30 mg of said herbal combination, and said housing contains at least 13.3 grams of the rehmannia glutinosa, at least 3.3 grams of the rheum tanguticum, and at least 3.3 grams of the lonicera japonica.

44. The treatment regimen of claim 42, wherein said multiple doses each include one or more inactive components for producing a cream, lotion, ointment or other topical formulation configured for direct application at a location of said skin wound.

45. The treatment regimen of claim 44, wherein said active component is diluted within said topical formulation between 1:50 and 1:500.

46. The treatment regimen of claim 42, wherein said multiple doses each include one or more inactive components for producing a shampoo, conditioner, or other topical scalp or hair treatment.

47. The treatment regimen of claim 42, wherein said active component comprises nanoparticulates of reduced average size to less than 450 nm.

48. The treatment regimen of claim 47, comprising one or more inactive ingredients combined with said nanoparticulates of said active component to produce a nanogel formulation configured for direct application at a location of said skin wound.

49. The treatment regimen of claim 42, wherein said active component comprises nanoparticulates of reduced average size to less than 350 nm.

50. The treatment regimen of claim 49, comprising one or more inactive ingredients combined with said nanoparticulates of said active component to produce a nanogel formulation configured for direct application at a location of said skin wound.

51. The treatment regimen of claim 42, wherein said active component comprises nanoparticulates of reduced average size to less than 250 nm.

52. The treatment regimen of claim 51, comprising one or more inactive ingredients combined with said nanoparticulates of said active component to produce a nanogel formulation configured for direct application at a location of said skin wound.

53. The treatment regimen of claim 42, comprising a combination of a methotrexate regimen with said multiple doses of said dosage composition for administration before, during, or after administration of said dosage composition, or combinations thereof.
